(19) United States
(12) Patent Application Publication
(10) Pub. No.: US 2023/0117235 A1
(43) Pub. Date: Apr. 20, 2023

(54) MACHINE LEARNING TECHNIQUES FOR PREDICTIVE RESPIRATORY QUALITY SCORE ASSIGNMENT

(71) Applicant: UnitedHealth Group Incorporated, Minnetonka, MN (US)

(72) Inventors: Jon Kevin Muse, Thompsons Station, TN (US); Rama S. Ravindranathan, Edison, NJ (US); Marilyn L. Gordon, Cherry Hill, NJ (US); Gregory J. Boss, Saginaw, MI (US)

(21) Appl. No.: 17/503,742

(22) Filed: Oct. 18, 2021

(51) Int. Cl. G16H 40/63; G06N 5/02

(52) U.S. Cl. CPC G16H 40/63; G06N 5/022

(57) ABSTRACT

Various embodiments of the present invention provide methods, apparatus, systems, computing devices, computing entities, and/or the like for performing predictive respiratory quality score assignment. Certain embodiments of the present invention utilize systems, methods, and computer program products that perform predictive respiratory quality score assignment using at least one of respiratory quality evaluation scoring machine learning models, explanation generation machine learning model, supplemental feature extraction machine learning model, and observed sensory data.

### BACKGROUND

Various embodiments of the present invention address technical challenges related to performing respiratory quality score assignment. Various embodiments of the present invention disclose innovative techniques for efficiently and effectively performing predictive respiratory quality score assignment using various predictive data analysis techniques.

### BRIEF SUMMARY

In general, various embodiments of the present invention provide methods, apparatus, systems, computing devices, computing entities, and/or the like for performing predictive respiratory quality score assignment. Certain embodiments of the present invention utilize systems, methods, and computer program products that perform predictive respiratory quality score assignment using at least one of respiratory quality evaluation machine learning model, explanation generation machine learning model, supplemental feature extraction machine learning model, and observed sensory data.

In accordance with one aspect, a method is provided. In one embodiment, the method comprises: identifying observed sensory data for a monitored individual; determining one or more input features for a respiratory quality evaluation machine learning model based at least in part on the observed sensory data; determining, based at least in part on the one or more input features, and using the respiratory quality evaluation machine learning model a respiratory quality score, wherein the respiratory quality score describes: (i) a predicted exertion phase level of a plurality of candidate exertion phase levels, and (ii) a respiratory quality level variance identifier of a plurality of respiratory quality level variance identifiers for the predicted exertion phase level; and performing one or more prediction-based actions based at least in part on the respiratory quality score

In accordance with another aspect, a computer product is provided. The computer program product may comprise at least one computer readable storage medium having computer-readable program code portions stored therein, the computer-readable program code portions comprising executable portions configured to: identify observed sensory data for a monitored individual; determine one or more input features for a respiratory quality evaluation machine learning model based at least in part on the observed sensory data; determine based at least in part on the one or more input features, and using the respiratory quality evaluation machine learning model a respiratory quality score, wherein the respiratory quality score describes: (i) a predicted exertion phase level of a plurality of candidate exertion phase levels, and (ii) a respiratory quality level variance identifier of a plurality of respiratory quality level variance identifiers for the predicted exertion phase level; and perform one or more prediction-based actions based at least in part on the respiratory quality score.

In accordance with yet another aspect, an apparatus comprising at least one processor and at least one memory including computer program code is provided. In one embodiment, the at least one memory and the computer program code may be configured to, with the processor, cause the apparatus to: identify observed sensory data for a monitored individual; determine one or more input features for a respiratory quality evaluation machine learning model based at least in part on the observed sensory data; determine based at least in part on the one or more input features, and using the respiratory quality evaluation machine learning model a respiratory quality score, wherein the respiratory quality score describes: (i) a predicted exertion phase level of a plurality of candidate exertion phase levels, and (ii) a respiratory quality level variance identifier of a plurality of respiratory quality level variance identifiers for the predicted exertion phase level; and perform, using the one or more processors, one or more prediction-based actions based at least in part on the respiratory quality score.

### BRIEF DESCRIPTION OF THE DRAWINGS

Having thus described the invention in general terms, reference will now be made to the accompanying drawings, which are not necessarily drawn to scale, and wherein:

FIG. 1 provides an exemplary overview of an architecture that can be used to practice embodiments of the present invention.

FIG. 2 provides an example predictive data analysis computing entity, in accordance with some embodiments discussed herein.

FIG. 3 provides an example client computing entity, in accordance with some embodiments discussed herein.

FIG. 4 is a flowchart diagram of an example process for performing predictive respiratory quality score assignment, in accordance with some embodiments discussed herein.

FIG. 5 is a flowchart diagram of an example process for determining a respiratory quality score based at least in part on one or more input features, in accordance with some embodiments discussed herein.

FIG. 6 provides an example user interface depicting one or more respiratory rating requests, in accordance with some embodiments discussed herein.

FIG. 7 is a flow chart diagram of an example process for performing one or more prediction-based actions based at least in part on a respiratory quality score, in accordance with some embodiments discussed herein.

FIG. 8 is a flowchart diagram of an example process for determining explanatory metadata for a respiratory quality score, in accordance with some embodiments discussed herein.

FIG. 9 is a flow chart diagram of an example process for performing one or more prediction-based actions based at least in part on a respiratory quality score, in accordance with some embodiments discussed herein.

FIG. 10 provides an example user interface depicting one or more user activity recommendation notifications, in accordance with some embodiments discussed herein.

FIG. 11 is a flow chart diagram of an example process for performing one or more prediction-based actions based at least in part on a respiratory quality score, in accordance with some embodiments discussed herein.

FIG. 12 is a flow chart diagram of an example process for performing one or more prediction-based actions based at least in part on a respiratory quality score, in accordance with some embodiments discussed herein.

### DETAILED DESCRIPTION

Various embodiments of the present invention now will be described more fully hereinafter with reference to the accompanying drawings, in which some, but not all, embodiments of the inventions are shown. Indeed, these inventions may be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated.

The terms “illustrative” and “exemplary” are used to be examples with no indication of quality level. Like numbers refer to like elements throughout. Moreover, while certain embodiments of the present invention are described with reference to predictive data analysis, one of ordinary skill in the art will recognize that the disclosed concepts can be used to perform other types of data analysis.

### I. Overview and Technical Improvements

Various embodiments of the present invention address technical challenges related to efficiently and effectively performing predicted respiratory quality score assignment based at least on observed sensory data for a monitored individual. The disclosed techniques improve the efficiency and effectiveness of respiratory quality score assignment using a respiratory quality evaluation machine learning model configured to generate a respiratory quality score, that describes a predicted exertion phase level and a respiratory quality level variance, based at least in part on one or more input features that is derived from observed sensory data for a monitored individual.

In some embodiments, respiratory quality evaluation machine learning model utilizes operations that may, in at least some embodiments, reduce or eliminate the need for computationally expensive training operations in order to generate the noted respiratory quality evaluation machine learning model. By reducing or eliminating the noted training operations, various embodiments of the present invention: (i) reduce or eliminate the computational operations needed for training and thus improves the computational efficiency of performing predictive respiratory quality score assignment, (ii) reduce or eliminate the need for storage resources to train/generate respiratory quality evaluation machine learning models and thus improves storage efficiency of performing predictive respiratory quality score assignment, and (iii) reduce or eliminate the need for transmitting extensive training data needed to generate respiratory quality evaluation machine learning models and thus improves transmission/network efficiency of performing predictive respiratory quality score assignment. Via the noted advantages, various embodiments of the present invention make substantial technical contributions to the fields of respiratory quality score assignment in particular and healthcare-related predictive data analysis in general.

An exemplary application of various embodiments of the present invention relates to proactively monitoring sensory data of asthma and chronic obstructive pulmonary disorder (COPD) patients (e.g., risk profiled patients) and determining in real-time, the best location, outdoor/indoor activities to be involved in, travel locations and other activities that will facilitate a better respiratory quality score for these patients. Breathing anomalies such as COPD is one of the leading causes of death. Generally, as understood, persons with asthma, COPD and other conditions have no accurate means to determine how the environment will impact them in their movement outdoors. Air quality indexes may not be sufficient as they may only provide a small subset of the data needed to help a person navigate day-to-day. In addition, some embodiments of the present invention enables respiratory therapy for patients unable to appear in person at a treatment center in that it offers live access to care personnel for real-time guidance. In some embodiments, the present invention may help a non-patient, athlete, and/or a typical individual to increase lung capacity safely or in a prescribed manner.

### II. Definitions

The term “observed sensory data” may refer to a data object received from one or more sensor devices about a current condition of a monitored individual. Examples of observed sensory data include blood oxygen (SpO2) level, heart rate, respiration rate, step count, body temperature, carbon dioxide (CO2) level, travel rate, and/or the like, where travel rate may describe a distance traveled by a monitored individual (e.g. measured in miles per hour and/or calculated by feet per minute). In some embodiments, observed sensory data may describe one or more data about a condition of a monitored individual to whom a predictive data analysis computing entity seeks to obtain one or more predicted recommended actions. For example, in some embodiments, observed sensory data may describe one or more data associated with respiration and/or breathing of a monitored individual. In some embodiments, the observed sensory data may comprise biometric data. As noted above, observed sensory data may be received from one or more sensor devices. For example, the predictive data analysis computing entity may receive blood oxygen (SpO2) level from a pulse oximeter. As another example, the predictive data analysis computing entity may receive step count data from an accelerometer. In some embodiments, observed sensory data may be received from one or more Internet of Things (IoT) devices.

The term “monitored individual” may refer to an individual whose biometric data, environmental conditions, location data, health record, physical condition, and/or the like are monitored using one or more monitoring devices (e.g., sensor devices) to help facilitate better respiratory quality score. For example, the monitored individual may be an individual with asthma or chronic obstructive pulmonary disorder (COPD) (e.g., at risk profiled patients). In some embodiments, vitals (e.g., blood oxygen (SpO2) level), environmental conditions, user activity and patterns of a monitored individual may be monitored using various monitoring devices (e.g., sensor devices) to help facilitate better respiratory quality score by determining and recommending the best location, best environment, user activities, and the like for the monitored individual. In some embodiments, a respiratory therapy plan may be generated and/or performed for a monitored individual. For example, live access to care personnel for real-time guidance for patients unable to appear in person at a treatment center. In some embodiments, the monitored individual may be a non-patient, athlete, and/or a typical individual whose biometric data is monitored to help increase lung capacity safely or in a prescribed manner.

The term “input features” may refer to a data construct provided to a respiratory quality evaluation machine learning model as part of the input for the respiratory quality evaluation machine learning model. In some embodiments, the input features may be determined based at least in part on the observed sensory data and may comprise adopting at least some (e.g., all) of the observed sensory features of the observed sensory data as the input features. In some embodiments, the input features may be used to train the respiratory quality evaluation machine model that is configured to predict a respiratory quality score for a monitored individual. In some embodiments, the input features may include a refined input feature. In some embodiments, the input features may be associated with respiration and/or other vitals of the monitored individual. As an example, a particular input feature may comprise a blood oxygen (SpO2) level for a monitored individual. As another example, a particular input feature may comprise a respiration rate of a monitored individual. As yet another example, a particular input feature may comprise a heart rate of a monitored individual. As a further example, a particular input feature may comprise a travel rate of a monitored individual. As yet a further example, a particular input feature may comprise a step count of a monitored individual. In some embodiments, determining the input features based at least in part on the observed sensory data comprises determining one or more engineered features for the monitored individual utilizing a supplemental feature extraction machine learning model, and determining the one or more input features based at least in part on the one or more engineered features and one or more observed sensory features defined by the observed sensory data.

The term “respiratory quality evaluation machine learning model” may refer to a data object that is configured to describe parameters, hyper-parameters, and/or defined operations of a model that is configured to generate a respiratory quality score for a monitored individual in relation to observed sensory data based at least in part on one or more input features. In some embodiments, the respiratory quality evaluation machine learning model is a supervised machine learning model (e.g., a neural network model) that is trained using labeled data, where the supervised machine learning model is configured to generate a respiratory quality score, where the respiratory quality score is configured to be used to determine a recommended prediction-based action for a monitored individual. In some embodiments, the respiratory quality evaluation machine learning model is an unsupervised machine learning model (e.g., a clustering model). In some embodiments, the inputs to a respiratory quality evaluation machine learning model include one or more input features, which may be a vector or a matrix. In some embodiments, the respiratory quality evaluation machine learning model may be configured to determine a plurality of candidate exertion phase levels for a monitored individual. In some embodiments, the plurality of candidate exertion phase levels may be indicative of a decline or positive responsiveness to drug and/or environmental change with respect to the monitored individual.

The term “respiratory quality score” may refer to a data object that is configured to describe a value that in turn describes an inferred respiratory satisfaction or distress of a monitored individual, and where the respiratory quality score may be indicative of real-time respiration/breathing quality of a monitored individual. In example embodiments, the respiratory quality score may be configured to be used to determine one or more recommended prediction-based actions for a monitored individual. In example embodiments, the respiratory quality score may describe a predicted exertion phase level of a plurality of candidate exertion phase levels and a respiratory quality level variance identifier of a plurality of respiratory quality level variance identifiers for the predicted exertion phase level. The respiratory quality score may be generated by a trained respiratory quality evaluation machine learning model by processing one or more input features for a corresponding monitored individual. Thus, a respiratory quality score may be an output of a machine learning model.

The term “predicted exertion phase level” may refer to a data construct that is configured to describe an inferred exertion phase level of a plurality of candidate exertion phase levels for a monitored individual with respect to respiratory satisfaction or distress of the monitored individual, where an exertion phase level may describe the level of effort to respire/breathe due to increased oxygen use. As an example, exertion phase 1 (level 1) may describe a sedentary (e.g., sleep) exertion phase. As another example, exertion phase 2 (level 2) may describe a low exertion phase. As yet another example, exertion phase 3 (level 3) may describe a transitional exertion phase, As a further example, exertion phase 4 (level 4) may describe a nominal exertion phase. As yet further example, exertion phase 5 (level 5) may describe a negatively impactful exertion phase. As an additional example, exertion phase 6 (level 6) may describe a dangerous impactful exertion phase. In example embodiments, a respiratory quality score may be defined based at least in part by a predicted exertion phase level, where the predicted exertion phase level may serve as a coarse indicator of a respiratory quality score. For example a respiratory quality score of 31 may describe a respiratory quality score defined by exertion phase 3. As another example, a respiratory quality score of 46, may describe a respiratory quality score defined by exertion phase 4. As yet another example, a respiratory quality score of 59, may describe a respiratory quality score defined by exertion phase 5. In some embodiments, the predicted exertion phase level may be associated with one or more detected activities, such as walking, running, and or the like. For example, a running activity may be associated with a higher exertion phase level relative to a walking activity.

The term “candidate exertion phase levels” may refer to a data construct that is configured to describe a range of abilities of a monitored individual with respect to respiratory satisfaction or distress of the monitored individual. For example, in some embodiments, the plurality of candidate exertion phase levels may describe a plurality of candidate exertion phase levels unique to a monitored individual, where an exertion phase level may describe the level of effort to respire/breathe due to increased oxygen use. In example embodiments, a plurality of candidate exertion phase levels for a monitored individual may comprise a sedentary exertion phase level, a low exertion phase level, a transitional exertion phase level, a nominal exertion phase level, a negatively impactful exertion phase level, and a dangerous impactful exertion phase level, where each exertion phase level may be characterized by one more defined biometric data, such as blood oxygen (SpO2) level, respiration rate, heart rate, step count, travel rate, and/or the like. In some embodiments, the plurality of candidate exertion phase levels may be determined based at least in part on a biometric timeseries data object and/or an activity timeseries data object, utilizing a machine learning model such as a respiratory quality evaluation machine learning model. In some embodiments, each exertion phase level of the plurality of candidate exertion phase levels may be associated with a plurality of exertion phase sub-levels.

The term “exertion phase hierarchy” may refer to a data construct that is configured to describe a plurality of sub-levels of an exertion phase level for a monitored individual, where each exertion phase sub-level may describe a respiratory satisfaction/distress level (e.g., comfort level) of the monitored individual with respect to the corresponding exertion phase level. In some embodiments, each exertion phase sub-level may be associated with a unique respiratory quality score. For example, in some embodiments, a transitional exertion phase may comprise (i) an exertion phase sub-level that is comfortable for the monitored individual, and associated with a respiratory quality score of 31 (ii) a transitional exertion phase sub-level that is acceptable to the monitored individual but is not very comfortable, and is associated with a respiratory quality score of 36, and (iii) a transitional exertion phase sub-level that is uncomfortable and unacceptable for the monitored individual, and is associated with a respiratory quality score of 39.

The term “respiratory quality level variance identifier” may refer to a data construct that is configured to define (e.g., precisely define) an exertion phase sub-level of a corresponding exertion phase level. For example in some embodiments, a respiratory quality score may be defined at least in part by a respiratory quality level variance identifier, where the respiratory quality level variance identifier may serve as a fine indicator of the respiratory quality score. For example a respiratory quality score of 31 may describe a respiratory quality score defined by a respiratory quality level variance of 1. As another example, a respiratory quality score of 36, may describe a respiratory quality score defined by a respiratory quality level variance of 6. As yet another example, a respiratory quality score of 49, may describe a respiratory quality score defined by a respiratory quality level variance of 9. In example embodiments, a respiratory quality level variance identifier may be used to correlate biometric data to real-time respiratory satisfaction or distress feeling of a monitored individual. For example, in some embodiments, a respiratory quality level variance identifier may be determined based at least in part on real-time patient responses to respiratory rating requests (e.g., surveys, queries, and/or the like), where, a respiratory rating request may comprise a request to a monitored individual to rate his or her real-time feeling of respiratory satisfaction or distress based at least in part on a respiratory evaluation scale, where a rating of 10 may be indicative of lowest respiratory satisfaction (e.g., highest distress) and a rating of 1 may be indicative of a highest respiratory satisfaction (e.g., lowest distress).

The term “explanatory feature” may refer to a data construct provided to an explanation generation machine learning model as part of the input for the explanation generation machine learning model. In some embodiments, one or more explanatory features may be used to train an explanation generation machine model that is configured to determine one or more explanatory metadata for a respiratory quality score for a monitored individual. In some embodiments, one or more explanatory features may be used to train an explanation generation machine learning model that is configured to determine one or more explanatory metadata for a plurality of respiratory quality scores relative to each other (e.g., difference between exertion phase sub-levels). For example, in some embodiments, an explanatory feature may describe one or more conditions that negatively impacts a respiratory quality score of the monitored individual. As another example, in some embodiments, an explanatory feature may describe one or more conditions that positively impacts a respiratory quality score of the monitored individual. In some embodiments, the one or more explanatory features may comprise one or more environmental condition features associated with an environmental condition. In some embodiments, explanatory features may include humidity, outdoor temperature, barometric pressure, air quality, and/or the like.

The term “explanatory metadata” may refer to a data object that is configured to describe one or more inferred reasons for one or more respiratory quality scores for a monitored individual, which may be generated by an explanation generation machine learning model. For example, in some embodiments, explanatory metadata may describe one or more inferred reasons for a particular respiratory quality score. As another example, in some embodiments, explanatory metadata may describe one or more inferred reasons for a deviation in a respiratory quality score relative to one or more historical respiratory quality scores. As yet another example, in some embodiments, explanatory metadata may describe one or more inferred reasons for a difference between two respiratory quality scores, where each respiratory quality score may be associated with the same exertion phase level or different exertion phase levels. The one or more inferred reasons may describe an impact of an explanatory feature on a respiratory quality score. For example, in some embodiments, a particular explanatory metadata may describe that outdoor temperature between 75 and 90 degrees negatively impacts the respiratory quality score of a monitored individual with respect to a particular exertion phase level. In some embodiments, an explanatory metadata is determined by performing one or more explanatory data analysis operation on one or more explanatory features, utilizing an explanation generation machine learning model.

The term “explanation generation machine learning model” may refer to a data object that is configured to describe parameters, hyper-parameters, and/or defined operations of a model that is configured to generate explanatory metadata for one or more respiratory quality scores of a monitored individual in relation to one or more exertion phase levels of a plurality of candidate exertion phase levels based at least in part one or more explanatory features. In some embodiments, the explanation generation machine learning model is a supervised machine learning model (e.g., a neural network model) that is trained using labeled data, where the supervised machine learning model is configured to generate a predicted explanatory metadata, where the predicted explanatory metadata is configured to be used to determine a recommended prediction-based action for a monitored individual. In some embodiments, the explanation generation machine learning model is an unsupervised machine learning model (e.g., a clustering model). In some embodiments, the inputs to an explanation generation machine learning model include one or more explanatory features, which may be a vector or a matrix.

The term “environmental condition features” may refer to an electronically-stored data construct that is configured to describe data objects captured by one or more monitoring devices and/or electronic devices (e.g., one or more mobile devices, Internet of Things (IoT) devices, Bluetooth Low Energy (BLE) devices, and/or the like) that describe one or more physical phenomena related to an environment of interest (e.g., environment of a monitored individual). The environmental condition features may be captured by monitoring devices and/or electronic devices that include one or more environmental sensor devices. Examples of environmental condition features may include humidity, outdoor temperature, barometric pressure, air quality, and/or the like.

The term “supplemental feature extraction machine learning model” may refer to a data object that is configured to describe parameters, hyper-parameters, and/or defined operations of a model that is configured to generate one or more engineered features based at least in part on observed sensory data of a monitored individual. In some embodiments, the supplemental feature extraction machine learning model is a supervised machine learning model (e.g., a neural network model) that is trained using labeled data, where the supervised machine learning model is configured to generate one or more engineered features, where the one or more engineered features is configured to be used to determine input features which are in turn configured to be used to determine a recommended prediction-based action for a monitored individual. In some embodiments, the supplemental feature extraction machine learning model is an unsupervised machine learning model (e.g., a clustering model). In some embodiments, the inputs to a supplemental feature extraction machine learning model include observed sensory data, which may be a vector or a matrix.

The term “supplemental oxygen use likelihood indicator” may refer to a data construct that is configured to describe an estimated likelihood of an occurrence of supplemental oxygen intake for a monitored individual, where supplemental oxygen intake may describe an event where a monitored individual receives supplemental oxygen (e.g., portable oxygen). For example, supplemental oxygen use likelihood indicator may describe that a monitored individual recently received supplemental oxygen and/or is currently receiving supplemental oxygen. In some embodiments, supplemental oxygen use likelihood indicator may be configured to be utilized, at least in part, for determining shifts in the plurality of candidate exertion phase levels for a monitored individual.

The term “machine learning model” may refer to a data object that describes parameters, hyper-parameters, defined operations, and/or defined mappings of a model that is configured to process one or more prediction input values in accordance with one or more trained parameters of the machine learning models in order to generate a prediction. An example of a machine learning model is a mathematically derived algorithm (MDA). An MDA may comprise any algorithm trained using training data to predict one or more outcome variables. Without limitation, an MDA, as used herein, may comprise machine learning frameworks including neural networks, support vector machines, gradient boosts, Markov models, adaptive Bayesian techniques, and statistical models (e.g., timeseries-based forecast models such as autoregressive models, autoregressive moving average models, and/or an autoregressive integrating moving average models). Additionally, and without limitation, an MDA, as used in the singular, may include ensembles using multiple machine learning and/or statistical techniques.

The term “detected activity” may refer to a data construct that is configured to describe an activity associated with a monitored individual. Examples of detected activities may include running, sleeping, walking, climbing, resting, and/or the like. In some embodiments, a detected activity may be determined based at least in part on activity condition data received from one or more activity sensor devices. For example, in some embodiments a detected activity may be determined based at least in part on biometric data. In some embodiments, a respiratory quality score may be associated with a detected activity. In some embodiments, each detected activity of a monitored individual may be associated with an exertion phase level of a plurality of candidate exertion phase levels of the monitored individual. In some embodiments, a detected activity may be associated with a detected activity type, where a detected activity type may refer to an electronically-stored data construct that is configured to describe an activity category (e.g., a category of one or more activities characterized by defined parameters). For example, a detected activity type may describe one or more activities associated with high intensity (e.g., intense exercise). As another example, a detected activity type may describe one or more activities associated with low intensity (e.g., slow walk).

### III. Computer Program Products, Methods, and Computing Entities

Embodiments of the present invention may be implemented in various ways, including as computer program products that comprise articles of manufacture. Such computer program products may include one or more software components including, for example, software objects, methods, data structures, or the like. A software component may be coded in any of a variety of programming languages. An illustrative programming language may be a lower-level programming language such as an assembly language associated with a particular hardware architecture and/or operating system platform. A software component comprising assembly language instructions may require conversion into executable machine code by an assembler prior to execution by the hardware architecture and/or platform. Another example programming language may be a higher-level programming language that may be portable across multiple architectures. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query or search language, and/or a report writing language. In one or more example embodiments, a software component comprising instructions in one of the foregoing examples of programming languages may be executed directly by an operating system or other software component without having to be first transformed into another form. A software component may be stored as a file or other data storage construct. Software components of a similar type or functionally related may be stored together such as, for example, in a particular directory, folder, or library. Software components may be static (e.g., pre-established or fixed) or dynamic (e.g., created or modified at the time of execution).

A computer program product may include a non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media (including volatile and non-volatile media).

In one embodiment, a non-volatile computer-readable storage medium may include a floppy disk, flexible disk, hard disk, solid-state storage (SSS) (e.g., a solid state drive (SSD), solid state card (SSC), solid state module (SSM), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In one embodiment, a volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for or used in addition to the computer-readable storage media described above.

As should be appreciated, various embodiments of the present invention may also be implemented as methods, apparatus, systems, computing devices, computing entities, and/or the like. As such, embodiments of the present invention may take the form of an apparatus, system, computing device, computing entity, and/or the like, executing instructions stored on a computer-readable storage medium to perform certain steps or operations. Thus, embodiments of the present invention may also take the form of an entirely hardware embodiment, an entirely computer program product embodiment, and/or an embodiment that comprises a combination of computer program products and hardware performing certain steps or operations.

Embodiments of the present invention are described below with reference to block diagrams and flowchart illustrations. Thus, it should be understood that each block of the block diagrams and flowchart illustrations may be implemented in the form of a computer program product, an entirely hardware embodiment, a combination of hardware and computer program products, and/or apparatus, systems, computing devices, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some exemplary embodiments, retrieval, loading, and/or execution may be performed in parallel such that multiple instructions are retrieved, loaded, and/or executed together. Thus, such embodiments can produce specifically-configured machines performing the steps or operations specified in the block diagrams and flowchart illustrations. Accordingly, the block diagrams and flowchart illustrations support various combinations of embodiments for performing the specified instructions, operations, or steps.

### IV. Exemplary System Architecture

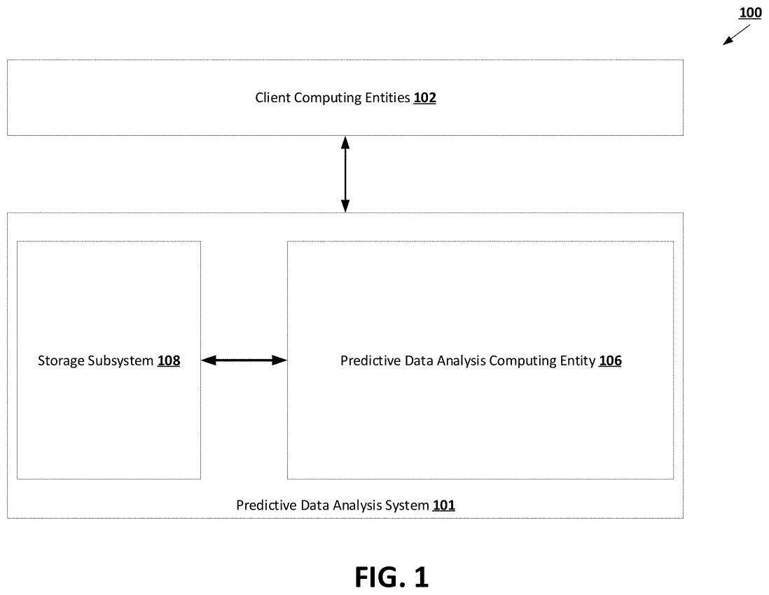

FIG. 1 is a schematic diagram of an example architecture 100 for performing predictive data analysis. The architecture 100 includes a predictive data analysis system 101 configured to generate predictive outputs that lead to performing one or more predictions. In some embodiments, the predictive data analysis system 101 may be configured to receive predictive data analysis requests from client computing entities 102, process the predictive data analysis requests to generate predictions, provide the generated predictions to the client computing entities 102, and automatically perform prediction-based actions based at least in part on the generated predictions.

An example of a prediction-based action that can be performed using the predictive data analysis system 101 is a request for generating a respiratory quality score for a monitored individual and recommending best situations (e.g., location, environment, activities, and/or the like that will facilitate a better respiratory quality score) for the monitored individual based at least in part on the respiratory quality score. Recommending best situations for a monitored individual plays an important role in medical and insurance fields. For example, it helps facilitate a better respiratory satisfaction (particularly in risk profiled patients such as those with asthma and chronic obstructive pulmonary disorder (COPD)). Thus recommending best situations based at least in part on respiratory quality score helps improve a patient's health and enables better treatment options.

In some embodiments, predictive data analysis system 101 may communicate with at least one of the client computing entities 102 using one or more communication networks. Examples of communication networks include any wired or wireless communication networks including, for example, a wired or wireless local area network (LAN), personal area network (PAN), metropolitan area network (MAN), wide area network (WAN), or the like, as well as any hardware, software and/or firmware required to implement it (such as, e.g., network routers, and/or the like).

The predictive data analysis system 101 may include a predictive data analysis computing entity 106 and a storage subsystem 108. The predictive data analysis computing entity 106 may be configured to receive predictive data analysis requests from one or more client computing entities 102, process the predictive data analysis requests to generate predictions corresponding to the predictive data analysis requests, provide the generated predictions to the client computing entities 102, and automatically perform prediction-based actions based at least in part on the generated predictions.

The storage subsystem 108 may be configured to store input data used by the predictive data analysis computing entity 106 to perform predictive data analysis, as well as model definition data used by the predictive data analysis computing entity 106 to perform various predictive data analysis tasks. The storage subsystem 108 may include one or more storage units, such as multiple distributed storage units that are connected through a computer network. Each storage unit in the storage subsystem 108 may store at least one of one or more data assets and/or one or more data about the computed properties of one or more data assets. Moreover, each storage unit in the storage subsystem 108 may include one or more non-volatile storage or memory media including, but not limited to, hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

### Exemplary Predictive Data Analysis Computing Entity

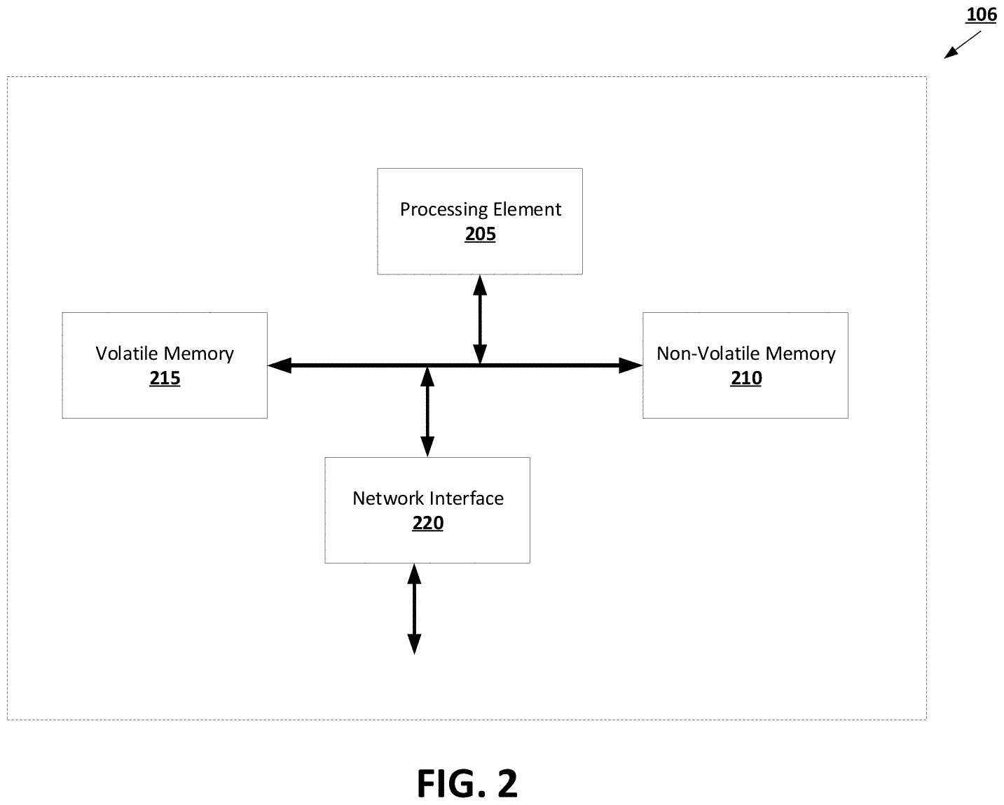

FIG. 2 provides a schematic of a predictive data analysis computing entity 106 according to one embodiment of the present invention. In general, the terms computing entity, computer, entity, device, system, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. Such functions, operations, and/or processes may include, for example, transmitting, receiving, operating on, processing, displaying, storing, determining, creating/generating, monitoring, evaluating, comparing, and/or similar terms used herein, interchangeably. In one embodiment, these functions, operations, and/or processes can be performed on data, content, information, and/or similar terms used herein, interchangeably.

As indicated, in one embodiment, the predictive data analysis computing entity 106 may also include one or more communications interfaces 220 for communicating with various computing entities, such as by communicating data, content, information, and/or similar terms used herein, interchangeably, that can be transmitted, received, operated on, processed, displayed, stored, and/or the like.

As shown in FIG. 2, in one embodiment, the predictive data analysis computing entity 106 may include, or be in communication with, one or more processing elements 205 (also referred to as processors, processing circuitry, and/or similar terms used herein, interchangeably) that communicate with other elements within the predictive data analysis computing entity 106 via a bus, for example. As will be understood, the processing element 205 may be embodied in a number of different ways.

For example, the processing element 205 may be embodied as one or more complex programmable logic devices (CPLDs), microprocessors, multi-core processors, coprocessing entities, application-specific instruction-set processors (ASIPs), microcontrollers, and/or controllers. Further, the processing element 205 may be embodied as one or more other processing devices or circuitry. The term circuitry may refer to an entirely hardware embodiment or a combination of hardware and computer program products. Thus, the processing element 205 may be embodied as integrated circuits, application specific integrated circuits (ASICs), field programmable gate arrays (FPGAs), programmable logic arrays (PLAs), hardware accelerators, other circuitry, and/or the like.

As will therefore be understood, the processing element 205 may be configured for a particular use or configured to execute instructions stored in volatile or non-volatile media or otherwise accessible to the processing element 205. As such, whether configured by hardware or computer program products, or by a combination thereof, the processing element 205 may be capable of performing steps or operations according to embodiments of the present invention when configured accordingly.

In one embodiment, the predictive data analysis computing entity 106 may further include, or be in communication with, non-volatile media (also referred to as non-volatile storage, memory, memory storage, memory circuitry and/or similar terms used herein, interchangeably).

In one embodiment, the non-volatile storage or memory may include one or more non-volatile storage or memory media 210, including, but not limited to, hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

As will be recognized, the non-volatile storage or memory media may store databases, database instances, database management systems, data, applications, programs, program modules, scripts, source code, object code, byte code, compiled code, interpreted code, machine code, executable instructions, and/or the like. The term database, database instance, database management system, and/or similar terms used herein, interchangeably, may refer to a collection of records or data that is stored in a computer-readable storage medium using one or more database models, such as a hierarchical database model, network model, relational model, entity—relationship model, object model, document model, semantic model, graph model, and/or the like.

In one embodiment, the predictive data analysis computing entity 106 may further include, or be in communication with, volatile media (also referred to as volatile storage, memory, memory storage, memory circuitry and/or similar terms used herein, interchangeably). In one embodiment, the volatile storage or memory may also include one or more volatile storage or memory media 215, including, but not limited to, RAM, DRAM, SRAM, FPM DRAM, EDO DRAM, SDRAM, DDR SDRAM, DDR2SDRAM, DDR3 SDRAM, RDRAM, TTRAM, T-RAM, Z-RAM, RIMM, DIMM, SIMM, VRAM, cache memory, register memory, and/or the like.

As will be recognized, the volatile storage or memory media may be used to store at least portions of the databases, database instances, database management systems, data, applications, programs, program modules, scripts, source code, object code, byte code, compiled code, interpreted code, machine code, executable instructions, and/or the like being executed by, for example, the processing element 205. Thus, the databases, database instances, database management systems, data, applications, programs, program modules, scripts, source code, object code, byte code, compiled code, interpreted code, machine code, executable instructions, and/or the like may be used to control certain aspects of the operation of the predictive data analysis computing entity 106 with the assistance of the processing element 205 and operating system.

As indicated, in one embodiment, the predictive data analysis computing entity 106 may also include one or more communications interfaces 220 for communicating with various computing entities, such as by communicating data, content, information, and/or similar terms used herein, interchangeably, that can be transmitted, received, operated on, processed, displayed, stored, and/or the like. Such communication may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. Similarly, the predictive data analysis computing entity 106 may be configured to communicate via wireless external communication networks using any of a variety of protocols, such as general packet radio service (GPRS), Universal Mobile Telecommunications System (UMTS), Code Division Multiple Access 2000 (CDMA2000), CDMA2000 1X (1xRTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.11 (Wi-Fi), Wi-Fi Direct, 802.16 (WiMAX), ultra-wideband (UWB), infrared (IR) protocols, near field communication (NFC) protocols, Wibree, Bluetooth protocols, wireless universal serial bus (USB) protocols, and/or any other wireless protocol.

Although not shown, the predictive data analysis computing entity 106 may include, or be in communication with, one or more input elements, such as a keyboard input, a mouse input, a touch screen/display input, motion input, movement input, audio input, pointing device input, joystick input, keypad input, and/or the like. The predictive data analysis computing entity 106 may also include, or be in communication with, one or more output elements (not shown), such as audio output, video output, screen/display output, motion output, movement output, and/or the like.

### Exemplary Client Computing Entity

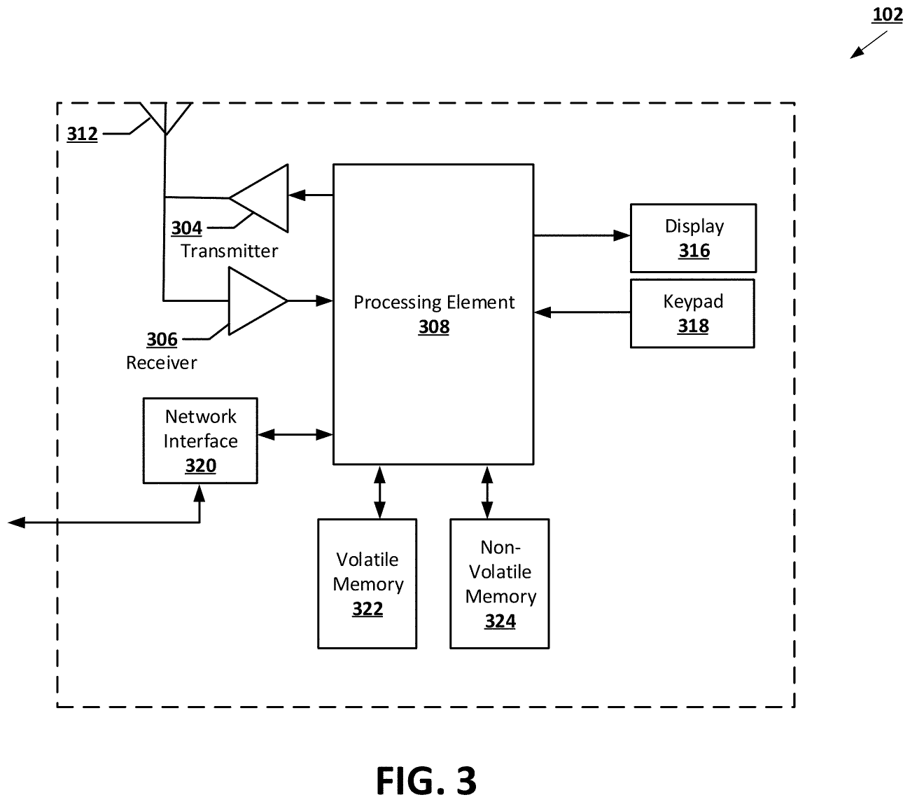

FIG. 3 provides an illustrative schematic representative of a client computing entity 102 that can be used in conjunction with embodiments of the present invention. In general, the terms device, system, computing entity, entity, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. Client computing entities 102 can be operated by various parties. As shown in FIG. 3, the client computing entity 102 can include an antenna 312, a transmitter 304 (e.g., radio), a receiver 306 (e.g., radio), and a processing element 308 (e.g., CPLDs, microprocessors, multi-core processors, coprocessing entities, ASIPs, microcontrollers, and/or controllers) that provides signals to and receives signals from the transmitter 304 and receiver 306, correspondingly.

The signals provided to and received from the transmitter 304 and the receiver 306, correspondingly, may include signaling information/data in accordance with air interface standards of applicable wireless systems. In this regard, the client computing entity 102 may be capable of operating with one or more air interface standards, communication protocols, modulation types, and access types. More particularly, the client computing entity 102 may operate in accordance with any of a number of wireless communication standards and protocols, such as those described above with regard to the predictive data analysis computing entity 106. In a particular embodiment, the client computing entity 102 may operate in accordance with multiple wireless communication standards and protocols, such as UMTS, CDMA2000, 1xRTT, WCDMA, GSM, EDGE, TD-SCDMA, LTE, E-UTRAN, EVDO, HSPA, HSDPA, Wi-Fi, Wi-Fi Direct, WiMAX, UWB, IR, NFC, Bluetooth, USB, and/or the like. Similarly, the client computing entity 102 may operate in accordance with multiple wired communication standards and protocols, such as those described above with regard to the predictive data analysis computing entity 106 via a network interface 320.

Via these communication standards and protocols, the client computing entity 102 can communicate with various other entities using concepts, such as Unstructured Supplementary Service Data (USSD), Short Message Service (SMS), Multimedia Messaging Service (MMS), Dual-Tone Multi-Frequency Signaling (DTMF), and/or Subscriber Identity Module Dialer (SIM). The client computing entity 102 can also download changes, add-ons, and updates, for instance, to its firmware, software (e.g., including executable instructions, applications, program modules), and operating system.

According to one embodiment, the client computing entity 102 may include location determining aspects, devices, modules, functionalities, and/or similar words used herein interchangeably. For example, the client computing entity 102 may include outdoor positioning aspects, such as a location module adapted to acquire, for example, latitude, longitude, altitude, geocode, course, direction, heading, speed, universal time (UTC), date, and/or various other information/data. In one embodiment, the location module can acquire data, sometimes known as ephemeris data, by identifying the number of satellites in view and the relative positions of those satellites (e.g., using global positioning systems (GPS)). The satellites may be a variety of different satellites, including Low Earth Orbit (LEO) satellite systems, Department of Defense (DOD) satellite systems, the European Union Galileo positioning systems, the Chinese Compass navigation systems, Indian Regional Navigational satellite systems, and/or the like. This data can be collected using a variety of coordinate systems, such as the Decimal Degrees (DD); Degrees, Minutes, Seconds (DMS); Universal Transverse Mercator (UTM); Universal Polar Stereographic (UPS) coordinate systems; and/or the like. Alternatively, the location information/data can be determined by triangulating the client computing entity's 102 position in connection with a variety of other systems, including cellular towers, Wi-Fi access points, and/or the like. Similarly, the client computing entity 102 may include indoor positioning aspects, such as a location module adapted to acquire, for example, latitude, longitude, altitude, geocode, course, direction, heading, speed, time, date, and/or various other information/data. Some of the indoor systems may use various position or location technologies including RFID tags, indoor beacons or transmitters, Wi-Fi access points, cellular towers, nearby computing devices (e.g., smartphones, laptops) and/or the like. For instance, such technologies may include the iBeacons, Gimbal proximity beacons, Bluetooth Low Energy (BLE) transmitters, NFC transmitters, and/or the like. These indoor positioning aspects can be used in a variety of settings to determine the location of someone or something to within inches or centimeters.

The client computing entity 102 may also comprise a user interface (that can include a display 316 coupled to a processing element 308) and/or a user input interface (coupled to a processing element 308). For example, the user interface may be a user application, browser, user interface, and/or similar words used herein, interchangeably, executing on and/or accessible via the client computing entity 102 to interact with and/or cause display of information/data from the predictive data analysis computing entity 106, as described herein. The user input interface can comprise any of a number of devices or interfaces allowing the client computing entity 102 to receive data, such as a keypad 318 (hard or soft), a touch display, voice/speech or motion interfaces, or other input device. In embodiments including a keypad 318, the keypad 318 can include (or cause display of) the conventional numeric (0-9) and related keys (#, *), and other keys used for operating the client computing entity 102 and may include a full set of alphabetic keys or set of keys that may be activated to provide a full set of alphanumeric keys. In addition to providing input, the user input interface can be used, for example, to activate or deactivate certain functions, such as screen savers and/or sleep modes.

The client computing entity 102 can also include volatile storage or memory 322 and/or non-volatile storage or memory 324, which can be embedded and/or may be removable. For example, the non-volatile memory may be ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like. The volatile memory may be RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like. The volatile and non-volatile storage or memory can store databases, database instances, database management systems, data, applications, programs, program modules, scripts, source code, object code, byte code, compiled code, interpreted code, machine code, executable instructions, and/or the like to implement the functions of the client computing entity 102. As indicated, this may include a user application that is resident on the entity or accessible through a browser or other user interface for communicating with the predictive data analysis computing entity 106 and/or various other computing entities.

In another embodiment, the client computing entity 102 may include one or more components or functionalities that are the same or similar to those of the predictive data analysis computing entity 106, as described in greater detail above. As will be recognized, these architectures and descriptions are provided for exemplary purposes only and are not limiting to the various embodiments.

In various embodiments, the client computing entity 102 may be embodied as an artificial intelligence (AI) computing entity, such as an Amazon Echo, Amazon Echo Dot, Amazon Show, Google Home, and/or the like. Accordingly, the client computing entity 102 may be configured to provide and/or receive information/data from a user via an input/output mechanism, such as a display, a camera, a speaker, a voice-activated input, and/or the like. In certain embodiments, an AI computing entity may comprise one or more predefined and executable program algorithms stored within an onboard memory storage module, and/or accessible over a network. In various embodiments, the AI computing entity may be configured to retrieve and/or execute one or more of the predefined program algorithms upon the occurrence of a predefined trigger event.

### V. Exemplary System Operations

As described below, various embodiments of the present invention address technical challenges related to efficiently and effectively performing respiratory quality score assignment based at least in part on observed sensory data for a monitored individual. The disclosed techniques improve the efficiency and effectiveness of respiratory quality score assignment using a respiratory quality evaluation machine learning model that is configured to generate a respiratory quality score, that describes a predicted exertion phase level and a respiratory quality level variance, based at least in part on one or more input features that is derived from observed sensory data for a monitored individual. The respiratory quality evaluation machine learning model utilizes operations that may, in at least some embodiments, reduce or eliminate the need for computationally expensive training operations in order to generate the noted respiratory quality evaluation machine learning model.

By reducing or eliminating the noted training operations, various embodiments of the present invention: (i) reduce or eliminate the computational operations needed for training and thus improves the computational efficiency of performing predictive respiratory quality score assignment, (ii) reduce or eliminate the need for storage resources to train/generate respiratory quality evaluation machine learning models and thus improves storage efficiency of performing predictive respiratory quality score assignment, and (iii) reduce or eliminate the need for transmitting extensive training data needed to generate respiratory quality evaluation machine learning models and thus improves transmission/network efficiency of performing predictive respiratory quality score assignment. Via the noted advantages, various embodiments of the present invention make substantial technical contributions to the fields of respiratory quality score assignment in particular and healthcare-related predictive data analysis in general.

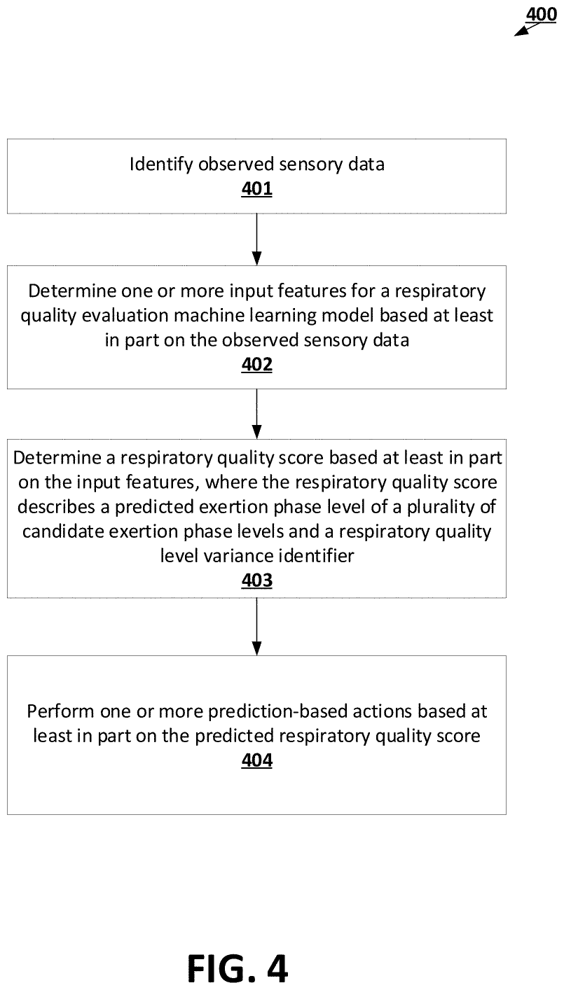

FIG. 4 is a flow chart diagram of an example process 400 for performing predictive respiratory quality score assignment, in accordance with some embodiments discussed herein. Via the various steps/operations of the process 400, the predictive data analysis computing entity 106 can relate respiratory quality score generated based at least in part on one or more input features to inferred respiratory satisfaction or distress of a monitored individual. While various embodiments of the present invention may describe performing multiple techniques using a singular computing entity, a person of ordinary skill in the relevant technology will recognize that each of the disclosed techniques can be performed by a separate computing entity. The process 400 will now be described with reference to the predictive data analysis computing entity 106 of the predictive data analysis system 101, as described above in relation to FIG. 1.

The process 400 begins at step/operation 401 when the predictive data analysis computing entity 106 identifies observed sensory data. In some embodiments, observed sensory data may describe one or more data about a condition of a monitored individual to whom the predictive data analysis computing entity 106 seeks to obtain one or more recommended prediction-based actions. For example, in some embodiments, the observed sensory data may describe one or more data associated with respiration and/or breathing of a monitored individual. In some embodiments, the observed sensory data may comprise biometric data. For example, in some embodiments, the observed sensory data may include blood oxygen (SpO2) level, heart rate, respiration rate, travel rate, step count, body temperature, carbon dioxide (CO2) level, and/or the like. In some embodiments, the observed sensory data may be a real-time observed sensory data. A person of ordinary skill in the relevant technology will recognize that the observed sensory data may comprise various other observed sensory data.

In some embodiments, the observed sensory data is received from one or more sensor devices. As an example, the predictive data analysis computing entity 106 may receive blood oxygen (SpO2) level from a pulse oximeter. As another example, the predictive data analysis computing entity 106 may receive step count data from an accelerometer. In some embodiments, the predictive data analysis computing entity 106 may receive observed sensory data from one or more Internet of Things (IoT) devices. In some embodiments, the predictive data analysis computing entity 106 may receive observed sensory data from one or more computing entities (e.g., client computing entity 102).

At step/operation 402, the predictive data analysis computing entity 106 determines one or more input features for a respiratory quality evaluation machine learning model based at least in part on the observed sensory data. In some embodiments, determining the one or more input features includes processing the observed sensory data utilizing a machine learning model. In some embodiments, the input features may be associated with respiration and/or other vitals of the monitored individual. As an example, a particular input feature may comprise a blood oxygen (SpO2) level of a monitored individual. As another example, a particular input feature may comprise a respiration rate of a monitored individual. As yet another example, a particular input feature may comprise a heart rate of a monitored individual. As further example, a particular input feature may comprise a travel rate of a monitored individual. As yet further example, a particular input feature may comprise a step count of a monitored individual.

In some embodiments, determining the one or more input features based at least in part on the observed sensory data comprises adopting at least some (e.g., all) of the observed sensory features of the observed sensory data as the input features. As an example, in some embodiments, the input features may comprise blood oxygen (SpO2) level and heart rate. As another example, in some embodiments, the input features may comprise blood oxygen (SpO2) level, heart rate, and step count. As yet another example, in some embodiments, the input features may comprise blood oxygen (SpO2) level, heart rate, respiration rate, step count, and travel rate. A person of ordinary skill in the relevant technology will recognize that the input features may comprise various other input features.

In some embodiments, determining the one or more input features comprise (i) determining, based at least in part on the observed sensory data and using a supplemental feature extraction machine learning model, one or more engineered features for the monitored individual, and (ii) determining the one or more input features based at least in part on the one or more engineered features and one or more observed sensory features defined by the observed sensory data. In some embodiments, the one or more engineered features may include a supplemental oxygen use likelihood indicator for the monitored individual, where a supplemental oxygen use likelihood indicator may describe an estimated likelihood of an occurrence of supplemental oxygen intake for a monitored individual, where supplemental oxygen intake may describe an event where a monitored individual receives supplemental oxygen (e.g., portable oxygen). For example, supplemental oxygen intake may describe that a monitored individual recently received supplemental oxygen and/or is currently receiving supplemental oxygen.

In some embodiments, the predictive data analysis computing entity 106 may be configured to monitor supplemental oxygen intake (and/or recommendation of supplemental oxygen) based at least in part on sensory data (e.g., SpO2, CO2, and the like). in some embodiments, monitoring supplemental oxygen intake may comprise determining whether a blood oxygen (SpO2) level measure satisfies an oxygen spike threshold and/or whether a carbon dioxide (CO2) level measure satisfies a carbon dioxide spike threshold. In the noted embodiments, a sudden increase in blood oxygen (SpO2) level and/or carbon dioxide (CO2) level may be indicative of supplemental oxygen intake. In some embodiments, monitoring supplemental oxygen intake may comprise determining a oxygen supply proximity measure, and determining whether the oxygen supply proximity measure satisfies a proximity measure threshold, where a oxygen supply proximity measure may describe a proximity of the monitored individual to a oxygen supply device (e.g., oxygen tank). In some embodiments, the oxygen supply proximity measure may be determined based at least in part on one or more sensor devices (e.g., Bluetooth and/or the like).

In some embodiments, monitoring supplemental oxygen intake may comprise transmitting one or more supplemental oxygen confirmation notifications to a computing entity associated with the monitored individual, where the supplemental oxygen confirmation notification may comprise a request for the monitored individual to confirm use/intake of supplemental oxygen. For example, in some embodiments, in response to determining that a blood oxygen (SpO2) level measure satisfies an oxygen spike threshold and/or a carbon dioxide (CO2) level measure satisfies a carbon dioxide spike threshold, and/or a oxygen supply proximity measure satisfies a oxygen supply proximity threshold, the predictive data analysis computing entity 106 may be configured to transmit a supplemental oxygen confirmation notification to a computing entity associated with the monitored individual.

At step/operation 403, the predictive data analysis computing entity 106 determines a respiratory quality score based at least in part on the input features, utilizing a respiratory quality evaluation machine learning model, where the respiratory quality score describes (i) a predicted exertion phase level of a plurality of candidate exertion phase levels, and (ii) a respiratory quality level variance identifier of a plurality of respiratory quality level variance identifiers for the predicted exertion phase level, where the respiratory quality score may be indicative of real-time respiration/breathing quality of a monitored individual. In some embodiments, the plurality of candidate exertion phase levels may be indicative of a decline or a positive responsiveness to drug and/or environmental change with respect to the monitored individual.

In some embodiments, the respiratory quality score may be associated with a detected activity having an activity type. In some embodiments, a detected activity may be determined based at least in part on biometric data (e.g., heart rate, pulse rate, and/or the like). In some embodiments, a detected activity may be determined based at least in part on user input. For example, in some embodiments, data indicative of an activity may be received from a computing entity associated with the monitored individual.

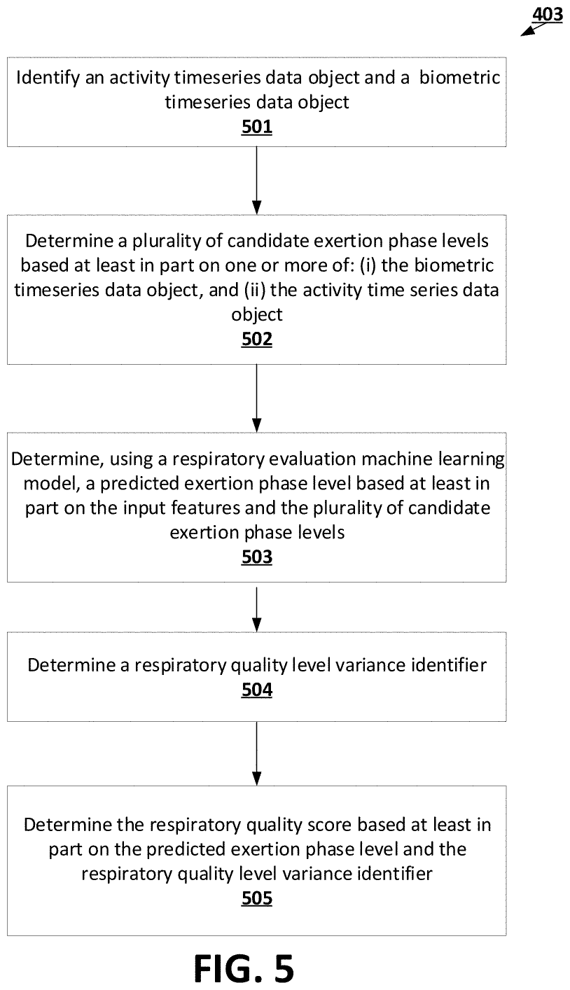

In some embodiments, the step/operation 403 may be performed in accordance with the process that is depicted in FIG. 5, which is an example process for determining a respiratory quality score, based at least in part on one or more input features, utilizing a respiratory quality evaluation machine learning model, where the respiratory quality score may describe a predicted exertion phase level of a plurality of candidate exertion phase levels and a respiratory quality level variance identifier of a plurality of quality level variance identifiers for the predicted exertion phase. The process begins at step/operation 501 when the predictive data analysis computing entity 106 identifies a biometric timeseries data object and an activity timeseries data object. In some embodiments, the biometric timeseries data object is a historic biometric timeseries data object that can be used to infer a plurality of candidate exertion phase levels. In some embodiments an activity timeseries data object is a historical activity timeseries data object that aligns temporally with the biometric timeseries data object that is a historic biometric timeseries data object.

In some embodiments, the activity timeseries data object describes one or more recorded user activity events for a monitored individual over one or more time periods, where each user activity event of the one or more recorded user activity events is associated with a timestamp. For example, in some embodiments, an activity timeseries data object may describe a recorded sleeping activity event of a monitored individual over one or more time periods. As another example, in some embodiments, an activity timeseries data object may describe a recorded running activity event of a monitored individual over one or more time windows. As yet another example, in some embodiments, an activity timeseries data object may describe a recorded resting activity event of a monitored individual over one or more time periods. As a further example, in some embodiments, an activity timeseries data object may describe a recorded sleeping activity, a recorded running activity, and/or a recorded resting activity event of a monitored individual over one or more time periods. A person of ordinary skill in the relevant technology will recognize that the activity timeseries data object may comprise various other recorded activity events.

In some embodiments, the data described by an activity timeseries data object is determined by using one or more activity sensor devices that are configured to monitor activity conditions of the monitored individual periodically or continuously over time and transmit the noted activity conditions to one or more computing entities, where the computing entities are configured to generate the activity timeseries data object based at least in part on the activity condition data that is received from the noted one or more activity sensors. In some embodiments, additionally or alternatively, the data described by the activity timeseries data object may be determined based at least in part on user input. For example, in some embodiments, the predictive data analysis computing entity 106 may receive, from one or more computing entities (e.g., a client computing entity 102), one or more data indicative of a user activity event of a monitored individual. In some embodiments, the activity timeseries data object is associated with an activity prediction window, where the activity prediction window may describe a time period across the one or more time periods across which the activity timeseries data object is calculated and across which one or more exertion phase levels may be inferred. For example, in some embodiments the activity prediction window may be over a period of one hour, two weeks, one month, and/or the like.

In some embodiments, a activity timeseries data object may be determined based at least in part on a user activity profile for a corresponding monitored individual, where the user activity profile may describe recorded user activity events of a corresponding prediction window. For example, in some embodiments, the activity timeseries data object may describe the normal activities of a monitored individual. In some embodiments, each user activity event may be associated with an activity severity level, where an activity severity level may describe the intensity level of the activity.

In some embodiments, a biometric timeseries data object describes one or more recorded biometric measures for a monitored individual over one or more time periods, where each biometric measure of the one or more recorded biometric measures is associated with a timestamp. For example, in some embodiments, the biometric timeseries data object may describe a recorded blood oxygen (SpO2) level of a monitored individual over one or more time periods. As another example, in some embodiments, the biometric timeseries data may describe a recorded respiration rate of a monitored individual over one or more time periods. As a further example, in some embodiments, the biometric timeseries data object may describe a recorded heart rate of a monitored individual over one or more time periods. As yet further example, the biometric timeseries data object may describe a travel rate of a monitored individual over one or more time periods. As an additional example, in some embodiments, the biometric timeseries data object may describe a step count of a monitored individual over one or more time periods. As yet an additional example, in some embodiments, the biometric timeseries data object may describe a recorded blood oxygen (SpO2) level, a recorded respiration rate, a recorded heart rate, a recorded travel rate, and/or a recorded step count of a monitored individual over one or more time periods.

In some embodiments, the data described by the biometric timeseries data object is determined by using one or more biometric sensor devices that are configured to monitor biometric conditions of the monitored individual periodically or continuously over time and transmit the noted biometric conditions to one or more computing entities, where the one or more computing entities are configured to generate the biometric timeseries data object based at least in part on the biometric condition data that is received from the noted one or more biometric sensors. In some embodiments, the biometric timeseries data object is associated with a biometric prediction window, where the biometric prediction window may describe a time period across the one or more time periods across which the biometric timeseries data object is calculated, and where the biometric prediction window temporally aligns with the activity prediction window.

In some embodiments, the predictive data analysis computing entity 106, may identify an environmental condition timeseries data object. In some embodiments, the environmental condition timeseries data object describes one more recorded environmental condition measures of an environment associated with a monitored individual over one or more time periods. For example, in some embodiments, the environmental condition timeseries data object may describe a recorded air quality of an environment of a monitored individual over one or more time periods. As another example, in some embodiments, the environmental condition timeseries data may describe a recorded humidity of an environment of a monitored individual over one or more time periods. As yet another example, in some embodiments, the environmental condition timeseries data object may describe a recorded outdoor temperature of an environment of a monitored individual over one or more time periods. As a further example, in some embodiments, the environmental condition timeseries data object may describe a recorded barometric pressure of an environment of a monitored individual over one or more time periods. As yet further example, the environmental condition timeseries data object may describe a recorded air quality, a recorded humidity, a recorded outdoor temperature, and/or a recorded barometric pressure of a monitored individual over one or more time periods.

In some embodiments, the data described by the environmental condition timeseries data object is determined by using one or more environmental condition sensor devices that are configured to monitor environmental conditions of the monitored individual periodically or continuously over time and transmit the noted environmental conditions to one or more computing entities, where the one or more computing entities are configured to generate the environmental condition timeseries data object based at least in part on the environmental condition data that is received from the noted one or more environmental sensor devices. In some embodiments, the environmental condition timeseries data object is associated with an environmental prediction window, where the environmental prediction window may describe a time period across the one or more time periods across which the environmental condition timeseries data object is calculated, and where the environmental prediction window temporally aligns with the activity prediction window.

At step/operation 502, the predictive data analysis computing entity 106 determines a plurality of candidate exertion phase levels based at least in part on one or more of: (i) the biometric timeseries data object, and the (ii) the activity time series data object. In some embodiments, determining the plurality of candidate exertion phase levels comprises analyzing the biometric timeseries data object with respect to the activity time series data object and/or the environmental condition timeseries data object. In some embodiments, determining the plurality of candidate exertion phase levels comprises correlating the biometric timeseries data object to one or more respiratory satisfaction or distress ratings of the monitored individual received from a computing entity.

In some embodiments, the plurality of candidate exertion phase levels comprise: (i) exertion phase 1 that describes a sedentary/sleep exertion phase, (ii) exertion phase 2 that describes a low exertion phase, (iii) exertion phase 3 that describes a transitional exertion phase, (iv) exertion phase 4 that describes a nominal exertion phase, (v) exertion phase 5 that describes a negatively exertion phase, and (vi) exertion phase 6 that describes a dangerous impactful exertion phase. In some embodiments, each exertion phase level of the one more exertion phase levels is characterized by one or more defined biometric data measure. In example embodiments, a sedentary/sleep exertion phase level may be characterized by average motionless SpO2 level, average respiration rate, average heart rate, and/or zero travel rate (e.g., no variance in location).

In some embodiments, a low exertion phase may be characterized by peak SpO2 levels, low step counts, low travel rate, average respiration rate, and/or average heart rate.

In example embodiments, a transitional exertion phase level may be characterized by minor reduced SpO2 levels relative to the low exertion phase level (e.g., below peak SpO2 levels), minor increased step counts relative to the low exertion phase level, minor increased travel rate relative to the low exertion phase level, slight increase in respiration rate relative to the low exertion phase level (e.g., above average respiration rate), and/or slight increase in heart rate relative to the low exertion phase level (e.g., above average heart rate). In some embodiments, the transitional phase level may serve as a trigger for the predictive data analysis computing entity 106 to begin specific pattern learning. In some embodiments, the transitional exertion phase may represent an ideal model for the monitored individual's normal exertion.

In example embodiments, a normal exertion phase level may be characterized by a reduced SpO2 level relative to the transitional exertion phase level (e.g., a linear pattern of reduced SpO2 levels), increase in step count relative to the transitional exertion phase level, increase in travel rate relative to the transitional exertion phase level, increase in respiration rate relative to the transitional exertion phase level, and/or increase in heart rate relative to the transitional exertion phase level. In some embodiments, the normal exertion phase level may represent the standard for maximum non-distressed breathing efficiency to facilitate determining the peak ability of the monitored individual. In some embodiments, the normal exertion phase level may represent the ideal pattern for safe increased exertion (e.g., exercise intensity).

In example embodiments, a negatively impactful exertion phase level may be characterized by a decrease in SpO2 level relative to the nominal exertion phase level (e.g., significant decrease in SpO2 level), increased respiration rate relative to the nominal exertion phase level (e.g., respiration that increases decline in SpO2 level during reduced step counts and reduced travel rate). In some embodiments, the SpO2 level associated with the impactful exertion phase may represent the detected lower limit of SpO2 for the monitored individual. In example embodiments, a dangerous impactful exertion phase level may be characterized by SpO2 level below medical standard and/or physician limits of SpO2 (e.g., below normal oxygen level that may be indicative of onset of hypoxemia).

In example embodiments, for each activity associated with the monitored individual, the predictive data analysis computing entity 106 may be configured to assign an exertion phase level of the plurality of candidate exertion phase levels. In some embodiments, the predictive data analysis computing entity 106 determines an impact likelihood measure for an activity based at least in part on the assigned exertion phase, where an impact likelihood measure for an activity may describe a likelihood that the activity will negatively impact the respiratory quality score of the monitored individual. In some embodiments, the impact likelihood measure may describe a predicted magnitude of the impact.

At step/operation 503, the predictive data analysis computing entity 106 determines, utilizing a respiratory quality evaluation machine learning model, a predicted exertion phase level based at least in part on the input features and the plurality of candidate exertion phase levels.

At step/operation 504, the predictive data analysis computing entity 106 determines a respiratory quality level variance identifier. In some embodiments, determining a respiratory quality level variance identifier comprises receiving a respiratory satisfaction/distress rating indicative of a respiratory quality level variance identifier, where the real-time respiratory satisfaction/distress rating may describe a real-time respiratory satisfaction or distress of a monitored individual with respect to one or more exertion phase levels of the plurality of candidate exertion phase levels associated with the monitored individual. In some embodiments, the predictive data analysis computing entity 106 may be configured to transmit one or more respiratory rating requests to a computing entity associated with the monitored individual. In the noted embodiments, transmitting the one or more respiratory rating requests may comprise generating user interface for one or more respiratory rating requests for display using a display device of the computing entity.

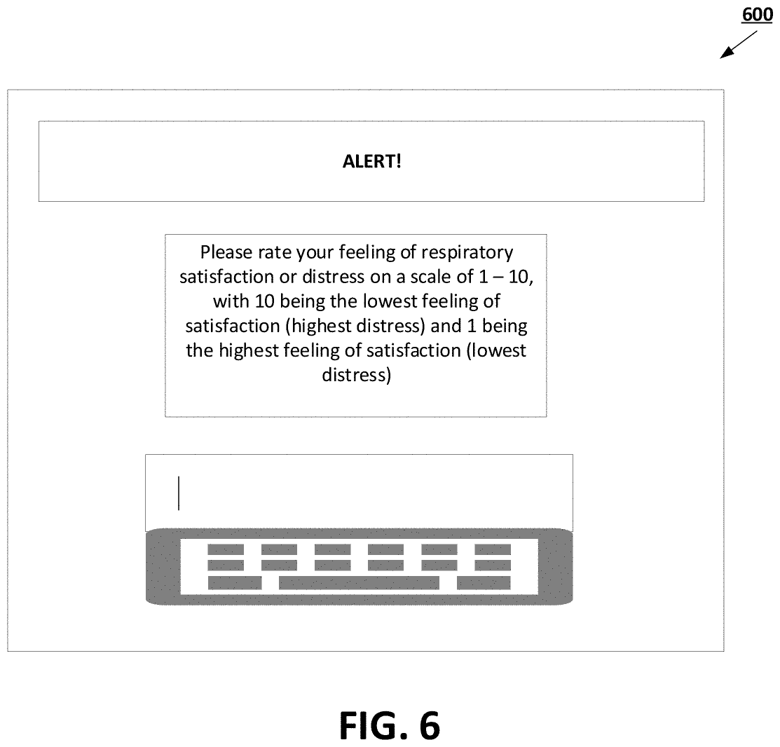

FIG. 6 provides an example user interface 600 depicting one or more respiratory rating requests, in accordance with some embodiments discussed herein. As depicted in FIG. 6, examples of a respiratory rating request may comprise a request to rate respiratory satisfaction or distress of a monitored individual based at least in part on a respiratory evaluation scale. In some embodiments, a respiratory evaluation scale may comprise a scale of 1-10, where a respiratory rating of 1 may describe a highest feeling of respiratory satisfaction (e.g., lowest distress) and a respiratory rating of 10 may describe a lowest feeling of respiratory satisfaction (e.g., highest distress). In some embodiments, the predictive data analysis computing entity 106 may display, using the display device of the computing entity, the respiratory evaluation scale.

Returning to FIG. 5, at step/operation 505, the predictive data analysis computing entity 106 determines the respiratory quality score based at least in part on the predicted exertion phase level and the respiratory quality level variance identifier. In some embodiments, the respiratory quality score may be characterized by one or more indicators, In some embodiments, a respiratory quality score may be defined by a coarse indicator and a fine indicator, where the course indicator may describe the corresponding predicted exertion phase level and the fine indicator may describe the corresponding real-time respiratory quality level variance identifier. As an example, in some embodiments, a respiratory quality score of 31 may describe a respiratory quality score defined by exertion phase 3 (e.g., transitional exertion phase) and a real time respiratory quality level variance identifier of 1, As another example, in some embodiments, a respiratory quality score of 36 may describe a respiratory quality score defined by exertion phase 3 and a real-time respiratory quality level variance identifier of 6, As yet another example, a predicted quality score of 39 may describe a respiratory quality score defined by exertion phase 3 and a real-time respiratory quality level variance identifier of 9. As a further example, a respiratory quality score of 46 may describe a respiratory quality score defined by exertion phase 4 (e.g., normal exertion phase) and a real-time respiratory quality level variance identifier of 6. As an additional example, a respiratory quality score of 49 may describe exertion phase 4 and a real-time respiratory quality level variance identifier of 9.

In some embodiments, an exertion phase hierarchy may define one or more defined exertion phase sub-levels for each candidate exertion phase level of the plurality of candidate exertion phase levels. In some embodiments, the respiratory quality evaluation machine learning model may be configured to generate a predicted exertion phase sub-level of the one or more defined exertion phase sub-levels for the predicted exertion phase level based at least in part on the one or more input features. In some embodiments, each exertion phase sub-level may be assigned a respiratory quality score of a plurality of respiratory quality scores based at least in part on the corresponding exertion phase levels and the corresponding respiratory quality level variance identifier.

In some embodiments, for each exertion phase hierarchy, the predictive data analysis computing entity 106 may be configured to determine one or more explanatory features for each exertion phase sub-level based at least in part on the associated respiratory quality score, utilizing an explanation generation machine learning model, where the one or more explanatory features may describe one or more conditions that impacts a respiratory quality score of the monitored individual. For example, in some embodiments, an explanatory feature may describe one or more conditions that negatively impacts a respiratory quality score of the monitored individual. As another example, in some embodiments, an explanatory feature may describe one or more conditions that positively impacts a respiratory quality score of the monitored individual.

Returning to FIG. 4, at step/operation 404, the predictive data analysis computing entity 106 performs one or more prediction-based actions based at least in part on the respiratory quality score. As noted above in some embodiments, the respiratory quality score may be associated with a detected activity having a detected activity type. In the noted embodiments performing the one or more prediction based actions based at least in part on the respiratory quality score may comprise: (i) determining a deviation measure based at least in part on the respiratory quality score and a historical respiratory quality score for the detected activity type, and (ii) performing one or more prediction-based actions based at least in part on the deviation measure. For example, in some embodiments, in response to determining that the deviation measure satisfies a deviation threshold, the predictive data analysis computing entity 106 may be configured to transmit a notification (e.g., alert, instructions, alarm, and/or the like) to a computing entity.

In some embodiments performing the one or more prediction-based actions based at least in part on the respiratory quality score comprises transmitting a recommended treatment notification for display on a user display of a computing entity (e.g., client computing entity 102). For example, in some embodiments, the recommended treatment notification may comprise additional atomizer treatment recommendation. In some embodiments, performing the one or more prediction-based actions based at least in part on the respiratory quality score comprises determining the likelihood of immobility of the monitored individual based at least in part on the respiratory quality score.

In some embodiments performing the one or more prediction-based actions based at least in part on the respiratory quality score comprises transmitting an emergency notification (e.g., alert, alarm, and/or the like) for display on a user display of a computing entity (e.g., client computing entity 102). In example embodiments, in response to determining that a respiratory quality score satisfies a respiratory quality score threshold, the predictive data analysis computing entity 106 may be configured to transmit an emergency notification (e.g., alert, and/or the like) to a computing entity. For example, the predictive data analysis computing entity 106 may transfer an emergency notification to a computing entity associated with the monitored individual. As another example, the predictive data analysis computing entity 106 may transfer an emergency notification to a computing entity associated with an individual associated with monitored individual (e.g., a friend, relative, physician, nurse, and or the like). In some embodiments the emergency notification may comprise a phone call.

In some embodiments performing the one or more prediction-based actions based at least in part on the respiratory quality score comprises determining an optimized respiratory therapy plan, and transmitting a recommended optimized respiratory therapy plan to a computing entity. In some embodiments determining the optimized respiratory therapy plan comprises determining one or more inferred respiratory patterns such as trends in biometric data (e.g., SpO2, CO2, and/or other vitals), and determining the optimized therapy plan based at least in part on the one or more inferred respiratory patterns. In some embodiments, the recommended optimized respiratory therapy plan may be configured such that it may be performed at any location and/or at any time. For example, in some embodiments, the predictive data analysis computing entity 106 may be configured to transmit one or more therapeutic notifications to a computing entity associated with the monitored individual, where the therapeutic notifications may comprise automated therapist instructions (e.g., automated voice feedback configured to instruct the monitored individual according to the respiratory therapist's direction).

In some embodiments, performing the one or more prediction-based actions based at least in part on the respiratory quality score comprise (i) determining whether a supplemental oxygen intake measure satisfies a supplemental oxygen intake threshold (e.g., excessive supplemental oxygen intake), and (ii) in response to determining that a supplemental oxygen intake measure satisfies a supplemental oxygen intake threshold, transmitting the excessive supplemental oxygen notifications (e.g., alert, alarm, and/or the like) for display using a display device of a computing entity (client computing entity 102), where the supplemental oxygen intake measure may be determined based at least in part on the respiratory quality score and and/or historical respiratory quality score and inferred patterns for the monitored individual. For example, in some embodiments, determining a predicted excessive supplemental oxygen intake may comprise analyzing blood oxygen (SpO2) level trend with respect to inspiration patterns. For example, in some embodiments a trending increase in SpO2 levels with lesser movement patterns of inspiration along with continued exertion may be indicative of excessive supplemental oxygen intake.

In some embodiments, in response to determining that a supplemental oxygen intake measure satisfies a supplemental oxygen intake threshold, the predictive data analysis computing entity 106 stores the excessive oxygen intake event in one or more databases (e.g., electronic health record/electronic medical record). In some embodiments, in response to determining that a supplemental oxygen intake measure satisfies a supplemental oxygen intake threshold, the predictive data analysis computing entity 106 may be configured to automatically reduce supplemental oxygen supply. For example, in some embodiments, the predictive data analysis computing entity 106 may be configured to automatically reduce the supply of supplemental oxygen, where observed SpO2 level satisfies a SpO2 level threshold (e.g., physician prescribed SpO2 limit) and/or observed CO2 level satisfies a CO2 threshold.

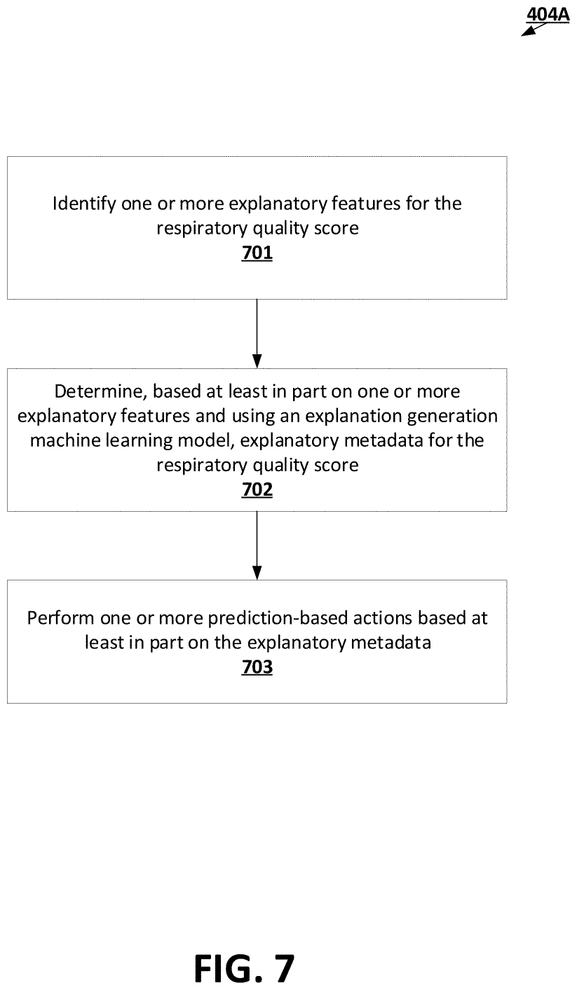

In some embodiments, the step/operation 404 may be performed in accordance with the process that is depicted in FIG. 7, which is an example process 404A for performing one or more prediction-based actions based at least in part on the respiratory quality score. The process begins at step/operation 701 when the predictive data analysis computing entity 106 identifies one or more explanatory features for the respiratory quality score. In some embodiments, the one or more explanatory features may comprise one or more environmental condition features associated with an environmental condition of the monitored individual.

At step/operation 702, the predictive data analysis computing entity 106 determines based at least in part on one or more explanatory features and using an explanation generation machine learning model, explanatory metadata for the respiratory quality score. In some embodiments, explanatory metadata may describe one or more inferred reasons for one or more respiratory quality scores for a monitored individual. For example, in some embodiments, the explanatory metadata may describe one or more inferred reasons for a particular respiratory quality score. As another example, in some embodiments, explanatory metadata may describe one more inferred reasons for a deviation in a respiratory quality score relative to one or more historical respiratory quality scores. As yet another example, in some embodiments, explanatory metadata may describe one or more inferred reasons for a difference between two respiratory quality scores, where each respiratory quality score may be associated with the same exertion phase level or different exertion phase levels. For example, in some embodiments, the predictive data analysis computing entity 106 may determine based at least in part on one or more explanatory metadata that the difference between a respiratory quality score of 31 and a respiratory quality score of 36 was due to increase in temperature and humidity.

As another example, the predictive data analysis computing entity 106 may determine based at least in part on one or more explanatory metadata that outdoor temperature between 75 and 90 degrees negatively impacts respiratory quality score of a monitored individual. In some embodiments, utilizing the explanation machine learning model, the tolerance and level of respiratory satisfaction of the monitored individual may be determined such that the monitored individual is not required to experience every possible respiratory quality score for modeling and prediction. In some embodiments, the predictive data analysis computing entity 106 may determine explanatory metadata that have a negative effect on the respiratory quality score of the monitored individual. In some embodiments, the predictive data analysis computing entity 106 may determine explanatory metadata that have a positive effect on the respiratory quality score of the monitored individual.

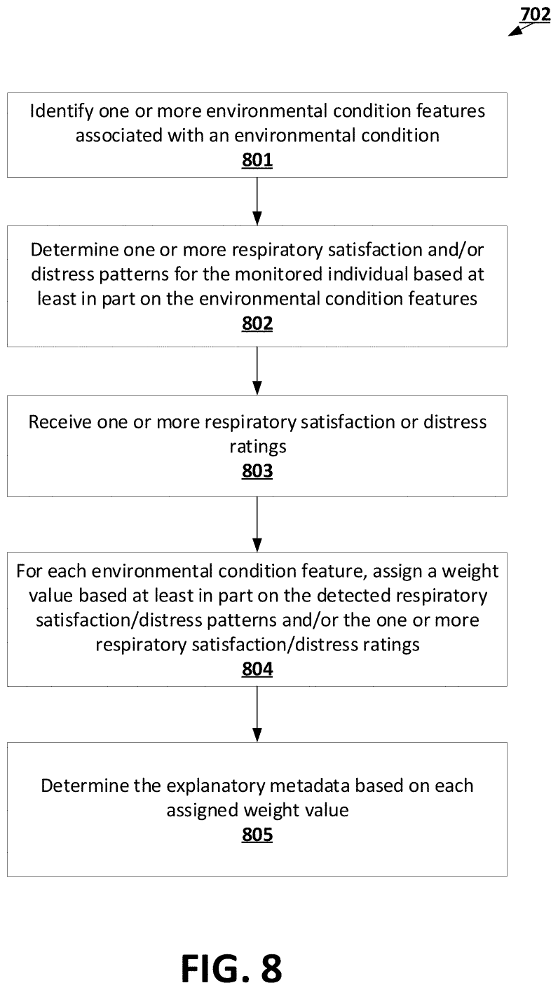

In some embodiments, the step/operation 702 may be performed in accordance with the process that is depicted in FIG. 8, which is an example process for determining, based at least in part on one or more explanatory features and using an explanation generation machine learning model, explanatory metadata for the respiratory quality score. The process begins at step/operation 801 when the predictive data analysis computing entity 106 identifies one or more environmental condition features associated with an environmental condition. In some embodiments, the predictive data analysis computing entity 106 may monitor environmental conditions of the immediate environmental surroundings of the monitored individual. For example, in some embodiments, the predictive data analysis computing entity 106 may monitor outdoor temperature, heat index, chill factor, humidity, barometric pressure, pollutants (e.g., pollutant that appear naturally or those detected due to locality specific occurrences) and/or the like. In some embodiments the predictive data analysis computing entity 106 may monitor environmental conditions utilizing Internet of Things (IoT) devices, wearable devices, mobile devices, and/or the like.

At step/operation 802, the predictive data analysis computing entity 106 determines one or more respiratory satisfaction and/or distress patterns for the monitored individual based at least in part on the environmental condition features. In some embodiments, determining the one or more respiratory satisfaction and/or distress patterns comprises analyzing the one or more environmental condition features with respect to the plurality of candidate exertion phase levels and/or the defined exertion phase sub-levels.

At step/operation 803, the predictive data analysis computing entity 106 receives one or more respiratory satisfaction or distress ratings from a computing entity (e.g., client computing entity 102) associated with the monitored individual. In some embodiments, the predictive data analysis computing entity 106 may transmit a notification (e.g., alert, survey, request and/or the like) to a computing entity associated with the monitored individual, where the notification comprise a request for the monitored individual to rate his or her feeling of respiratory satisfaction or distress, and where a rating of 10 may be indicative of a lowest feeling of respiratory satisfaction feeling (e.g., highest distress feeling) and a rating of 1 may be indicative of a highest feeling of respiratory satisfaction (e.g., lowest distress feeling).

At step/operation 804, for each detected environmental condition feature, the predictive data analysis computing entity 106 assigns a weight value (e.g. distress multiplier) based at least in part on the detected respiratory satisfaction/distress patterns and/or the one or more respiratory satisfaction/distress ratings. At step/operation 805, the predictive data analysis computing entity 106 determines the explanatory metadata based at least in part on each assigned weight value. In some embodiments, determining the explanatory metadata based at least in part on each assigned weight value comprises applying each assigned weight value to the corresponding environmental condition feature.

Returning to FIG. 7, at step/operation 703, the predictive data analysis computing entity 106 performs one or more prediction-based actions based at least in part on the explanatory metadata. In some embodiments performing the one more prediction-based actions based at least in part on explanatory metadata comprises (i) determining, based at least in part on the explanatory metadata, one or more user activity recommendations for the monitored individual, and (ii) performing the one or more prediction-based actions based at least in part on the one or more user activity recommendations. For example, in some embodiments, the predictive data analysis computing entity 106 may be configured to transmit a recommended user activity notification for display on a user display of a computing entity (e.g., client computing entity 102), where the recommended user activity notification may recommend an activity for the monitored individual.

In some embodiments performing the one more prediction-based actions based at least in part on the explanatory metadata comprises (i) determining, based at least in part on the explanatory metadata, one or more environmental condition modification recommendations for the monitored individual, and (ii) performing the one or more prediction-based actions based at least in part on the one or more environmental condition modification recommendations. In some embodiments, determining the one or more environmental condition modification recommendations for the monitored individual comprises determining the current location of the monitored individual and/or travel patterns (e.g., movement patterns) of the monitored individual. As an example, in some embodiments, the current location and/or travel patterns of a monitored individual may be determined based at least in part on one or more location sensor devices (e.g., Global Positioning System) and/or historical location data. As another example in some embodiments, the current location and/or travel pattern of a monitored individual may be determined based at least in part on a calendar of the monitored individual.

In some embodiments, performing the one or more prediction-based actions based at least in part on the one or more environmental condition modification recommendations may comprise transmitting a recommended environmental condition modification notification to a computing entity, where the recommended environmental condition modification notification may comprise a recommendation for a new environment (relative to a current environment and/or historical environment) for the monitored individual to occupy (e.g., based at least in part on the environmental conditions of the recommended environment and/or environmental conditions of the current environment and/or historical environment of the monitored individual). For example, in some embodiments, the recommended environmental modification notification may comprise a notification to take an alternate route due to poor air quality in the normal route taken by the monitored individual. As another example, in some embodiments, the recommended environmental modification notification may comprise a recommendation that the monitored individual consider staying indoors (e.g., at home) that particular day. As yet another example, in some embodiments, the recommended environmental condition modification notification may comprise a recommendation that the monitored individual receive supplemental oxygen if he or she intends to go on a hike. In some embodiments, the recommended environmental condition modification notification may be based at least in part on analysis of a current path of the monitored individual. For example, a recommended environmental condition modification notification may describe that it appears the monitored individual is on his or her way to a particular park and he or she should avoid the particular park due to poor environmental condition (e.g., air quality) of the particular park.

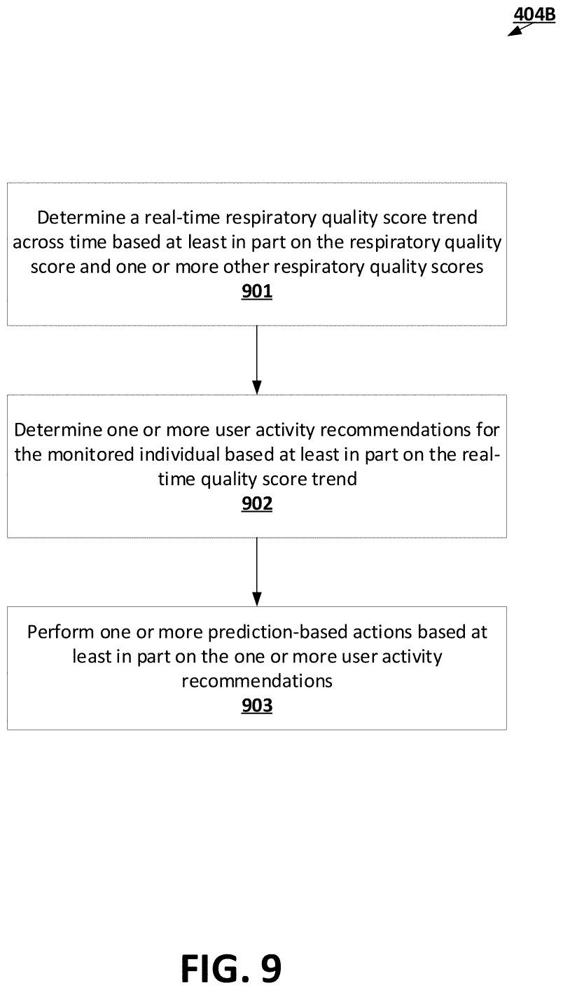

Returning to FIG. 4, in some embodiments, the step/operation 404 may be performed in accordance with the process that is depicted in FIG. 9, which is an example process 404B for performing one or more prediction-based actions based at least in part on the respiratory quality score. The process begins at step/operation 901 when the predictive data analysis computing entity 106 determines a real-time respiratory quality score trend across time based at least in part on the respiratory quality score and one or more other respiratory quality scores (e.g., historical respiratory quality scores), where a real-time respiratory quality score trend may describe a detected respiratory pattern of a monitored individual with respect to a plurality of respiratory quality scores. In some embodiments, determining the real-time respiratory quality score trend comprises analyzing supplemental oxygen intake. In some embodiments, determining the real-time respiratory quality score trend comprises analyzing step count pattern and associated deviation measure (e.g., increased step count to accomplish the same objective). In some embodiments, determining the real-time respiratory quality score trend comprises analyzing one or more exertion phase shifts (e.g., where less exertion is required to accomplish the same goals or more exertion is required to accomplish the same goals).

At step/operation 902, the predictive data analysis computing entity 106 determines one or more user activity recommendations for the monitored individual based at least in part on the real-time respiratory quality score trend. For example, the predictive data analysis computing entity 106 may determine based at least in part on analyzing the respiratory quality score trend that a running activity will negatively impact the respiratory quality score of the monitored individual and recommend that the monitored individual avoid running that particular day.

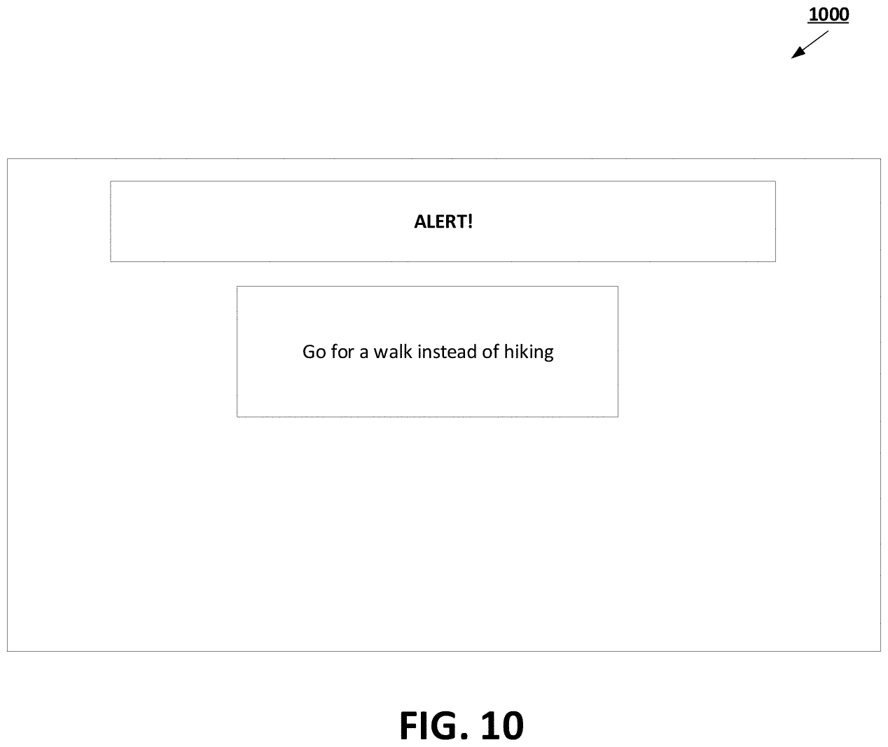

At step/operation 903, the predictive data analysis computing entity 106 performs one or more prediction-based actions based at least in part on the one or more user activity recommendations. In some embodiments, performing the one or more prediction-based actions includes generating a user interface data for one or more recommendation notifications for display using a display device of a computing entity. FIG. 10 provides an example user interface 1000 depicting one or more user activity recommendation notifications, in accordance with some embodiments discussed herein. As depicted in FIG. 10, examples of user activity recommendation notifications, may include automated instructions based at least in part on the one or more user activity recommendations. As an example, the automated instructions may comprise recommendation to go for walk instead of hiking.

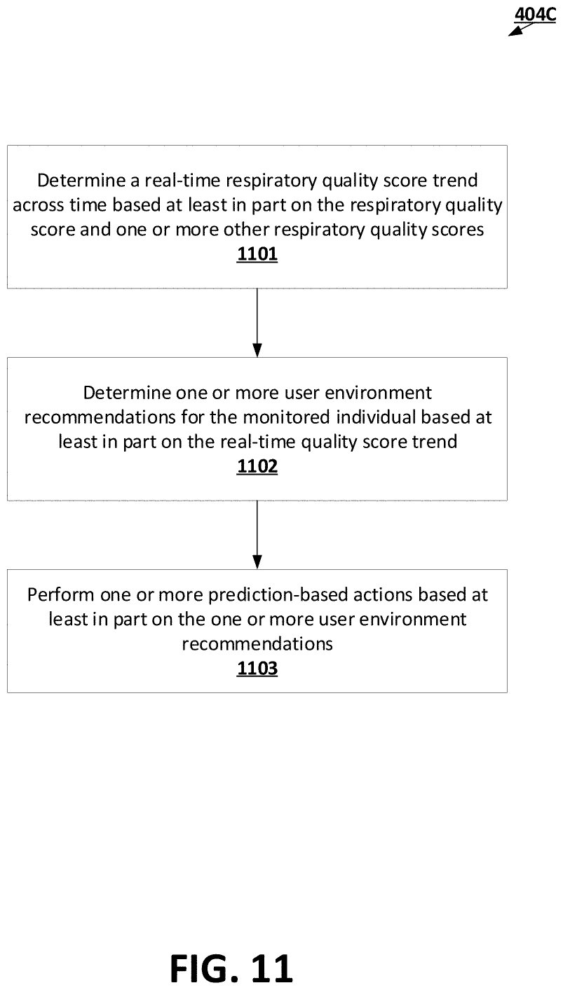

Returning to FIG. 4, in some embodiments, step/operation 404 may be performed in accordance with the process that is depicted in FIG. 11, which is an example process 404C for performing one or more prediction-based actions based at least in part on the respiratory quality score. The process begins at step/operation 1101 when the predictive data analysis computing entity 106 determines a real-time respiratory quality score trend across time based at least in part on the respiratory quality score and one or more other respiratory quality scores (e.g., historical respiratory quality scores). At step/operation 1102, the predictive data analysis computing entity 106 determines one or more user environment recommendations for the monitored individual based at least in part on the real-time respiratory quality score trend. At step/operation 1103, the predictive data analysis computing entity 106 performs one or more prediction-based actions based at least in part on the one or more user environment recommendations.

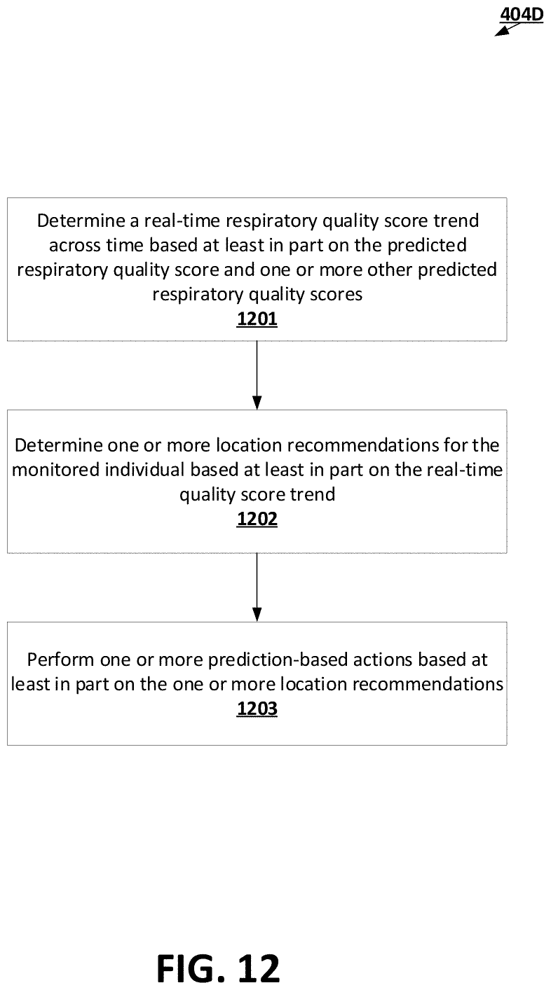

Returning to FIG. 4, in some embodiments, the step/operation 404 may be performed in accordance with the process that is depicted in FIG. 12, which is an example process 404D for performing one or more prediction-based actions based at least in part on the respiratory quality score. The process begins at step/operation 1201 when the predictive data analysis computing entity 106 determines a real-time respiratory quality score trend across time based at least in part on the respiratory quality score and one or more other respiratory quality scores (e.g., historical respiratory quality scores).

At step/operation 1202, the predictive data analysis computing entity 106 determines one or more location recommendations for the monitored individual based at least in part on the real-time respiratory quality score trend. In some embodiments determining the one or more location recommendation based at least in part on the real-time respiratory quality score trend includes analyzing respiratory quality score trends of one or more other monitored individuals. For example, in some embodiments, the predictive data analysis computing entity 106 determines a location recommendation (e.g., location to live) for the monitored individual through modeling of the respiratory quality scores associated with the monitored individual and the respiratory quality scores of one or more other monitored individuals, where the recommended location (e.g., new location) is associated with a higher likelihood of a better respiratory quality score for the monitored individual relative to the current location of the monitored individual. In some embodiments, determining the one or more location recommendations for the monitored individual based at least in part on the real-time respiratory quality score trend includes analyzing environmental conditions such as the seasons (e.g., fall, spring, summer, winter, and/or the like). At step/operation 1203, the predictive data analysis computing entity 106 performs one or more prediction-based actions based at least in part on the one or more location recommendations.

Accordingly, as described above, various embodiments of the present invention address technical challenges related to efficiently and effectively performing predicted respiratory quality score assignment based at least on observed sensory data for a monitored individual. The disclosed techniques improve the efficiency and effectiveness of respiratory quality score assignment using a respiratory quality evaluation machine learning model configured to generate a respiratory quality score, that describes a predicted exertion phase level and a respiratory quality level variance, based at least in part on one or more input features that is derived from observed sensory data for a monitored individual.

### VI. Conclusion

Many modifications and other embodiments will come to mind to one skilled in the art to which this disclosure pertains having the benefit of the teachings presented in the foregoing descriptions and the associated drawings. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

1. A computer-implemented method for predictive respiratory quality score assignment, the computer-implemented method comprising:
identifying, using one or more processors, observed sensory data for a monitored individual;
determining, using the one or more processors, one or more input features for a respiratory quality evaluation machine learning model based at least in part on the observed sensory data;
determining, using the one or more processors and the respiratory quality evaluation machine learning model, and based at least in part on the one or more input features, a respiratory quality score, wherein the respiratory quality score describes: (i) a predicted exertion phase level of a plurality of candidate exertion phase levels, and (ii) a respiratory quality level variance identifier of a plurality of respiratory quality level variance identifiers for the predicted exertion phase level; and
performing, using the one or more processors, one or more prediction-based actions based at least in part on the respiratory quality score.

2. The computer-implemented method of claim 1, wherein:
an exertion phase hierarchy defines one or more defined exertion phase sub-levels for each candidate exertion phase level, and
the respiratory quality evaluation machine learning model is configured to generate a predicted exertion phase sub-level of the one or more defined exertion phase sub-levels for the predicted exertion phase level based at least in part on the one or more input features.

3. The computer-implemented method of claim 1, wherein:
the respiratory quality score is associated with a detected activity having a detected activity type, and
the computer-implemented method further comprises:
determining a deviation measure based at least in part on the respiratory quality score and a historical respiratory quality score for the detected activity type; and
performing one or more second prediction-based actions based at least in part on the deviation measure.

4. The computer-implemented method of claim 1, further comprising:
identifying one or more explanatory features for the respiratory quality score;
determining, based at least in part on the one or more explanatory features and using an explanation generation machine learning model, explanatory metadata for the respiratory quality score; and
performing one or more third prediction-based actions based at least in part on the explanatory metadata.

5. The computer-implemented method of claim 4, wherein performing the one or more third prediction-based actions comprises:
determining, based at least in part on the explanatory metadata, one or more environmental condition modification recommendations for the monitored individual; and
performing the one or more prediction-based actions based at least in part on environmental condition modification recommendations.

6. The computer-implemented method of claim 4, wherein performing the one or more third prediction-based actions comprises:
determining, based at least in part on the explanatory metadata, one or more user activity recommendations for the monitored individual; and
performing the one or more prediction-based actions based at least in part on the one or more user activity recommendations.

7. The computer-implemented method of claim 1, wherein determining the one or more input features comprises:
determining, based at least in part on the observed sensory data and using a supplemental feature extraction machine learning model, one or more engineered features for the monitored individual; and
determining the one or more input features based at least in part on the one or more engineered features and one or more observed sensory features defined by the observed sensory data.

8. The computer-implemented method of claim 7, wherein the one or more engineered features is a supplemental oxygen use likelihood indicator for the monitored individual.

9. The computer-implemented method of claim 1, wherein performing the one or more prediction-based actions comprises:
determining a real-time respiratory quality score trend across time based at least in part on the respiratory quality score and one or more other respiratory quality scores;
determining, based at least in part on the real-time respiratory quality score trend, one or more user activity recommendations for the monitored individual; and
performing the one or more prediction-based actions based at least in part on the one or more user activity recommendations.

10. An apparatus for predictive respiratory quality score assignment, the apparatus comprising at least one processor and at least one memory including program code, the at least one memory and the program code configured to, with the processor, cause the apparatus to at least:
identify observed sensory data for a monitored individual;
determine one or more input features for a respiratory quality evaluation machine learning model based at least in part on the observed sensory data;
determine based at least in part on the one or more input features, and using the respiratory quality evaluation machine learning model a respiratory quality score, wherein the respiratory quality score describes: (i) a predicted exertion phase level of a plurality of candidate exertion phase levels, and (ii) a respiratory quality level variance identifier of a plurality of respiratory quality level variance identifiers for the predicted exertion phase level; and
perform one or more prediction-based actions based at least in part on the respiratory quality score.

11. The apparatus of claim 10, wherein:
an exertion phase hierarchy defines one or more defined exertion phase sub-levels for each candidate exertion phase level, and
the respiratory quality evaluation machine learning model is configured to generate a predicted exertion phase sub-level of the one or more defined exertion phase sub-levels for the predicted exertion phase level based at least in part on the one or more input features.

12. The apparatus of claim 10, wherein:
the respiratory quality score is associated with a detected activity having a detected activity type, and
the at least one memory and the program code are further configured to cause the apparatus to at least:
determine a deviation measure based at least in part on the respiratory quality score and a historical respiratory quality score for the detected activity type; and
perform one or more second prediction-based actions based at least in part on the deviation measure.

13. The apparatus of claim 10, wherein the at least one memory and the program code are further configured to cause the apparatus to at least:
identify one or more explanatory features for the respiratory quality score;
determine, based at least in part on the one or more explanatory features and using an explanation generation machine learning model, explanatory metadata for the respiratory quality score; and
perform one or more third prediction-based actions based at least in part on the explanatory metadata.

14. The apparatus of claim 13, wherein performing the one or more third prediction-based actions comprises:
determining, based at least in part on the explanatory metadata, one or more environmental condition modification recommendations for the monitored individual; and
performing the one or more prediction-based actions based at least in part on environmental condition modification recommendations.

15. The apparatus of claim 13, wherein performing the one or more third prediction-based actions comprises:
determining, based at least in part on the explanatory metadata, one or more user activity recommendations for the monitored individual; and
performing the one or more prediction-based actions based at least in part on the one or more user activity recommendations.

16. The apparatus of claim 10, wherein determining the one or more input features comprises:
determining, based at least in part on the observed sensory data and using a supplemental feature extraction machine learning model, one or more engineered features for the monitored individual; and
determining the one or more input features based at least in part on the one or more engineered features and one or more observed sensory features defined by the observed sensory data.

17. The apparatus of claim 16, wherein the one or more engineered features is a supplemental oxygen use likelihood indicator for the monitored individual.

18. The apparatus of claim 10, wherein performing the one or more prediction-based actions comprises:
determining a real-time respiratory quality score trend across time based at least in part on the respiratory quality score and one or more other respiratory quality scores;
determining, based at least in part on the real-time respiratory quality score trend, one or more user activity recommendations for the monitored individual; and
performing the one or more prediction-based actions based at least in part on the one or more user activity recommendations.

19. A computer program product for predictive respiratory quality score assignment, the computer program product comprising at least one non-transitory computer readable storage medium having computer-readable program code portions stored therein, the computer-readable program code portions configured to:
identify observed sensory data for a monitored individual;
determine one or more input features for a respiratory quality evaluation machine learning model based at least in part on the observed sensory data;
determine based at least in part on the one or more input features, and using the respiratory quality evaluation machine learning model a respiratory quality score, wherein the respiratory quality score describes: (i) a predicted exertion phase level of a plurality of candidate exertion phase levels, and (ii) a respiratory quality level variance identifier of a plurality of respiratory quality level variance identifiers for the predicted exertion phase level; and
perform one or more prediction-based actions based at least in part on the respiratory quality score.

20. The computer program product of claim 19, wherein:
an exertion phase hierarchy defines one or more defined exertion phase sub-levels for each candidate exertion phase level, and
the respiratory quality evaluation machine learning model is configured to generate a predicted exertion phase sub-level of the one or more defined exertion phase sub-levels for the predicted exertion phase level based at least in part on the one or more input features.
